SEQUENCE LISTING (44 sequences)

SEQ ID NO: 1 - DNA, 1095 bp, Homo sapiens
atgcatcctcaagtggtcatcttaagcctcatcctacatctggcagattctgtagctggttctgtaaaggttggtggagaggcaggtccatctgtcacactaccctgccactacagtggagctgtcacatccatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaatgacgactgttccaacgacaactgttccaacaacaatgagcattccaacgacaacgactgttctgacgacaatgactgtttcaacgacaacgagcgttccaacgacaacgagcattccaacaacaacaagtgttccagtgacaacaactgtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaa

SEQ ID NO: 2 - DNA, 1626 bp, Homo sapiens
agtgctattactgcatatgatgtaggtttagttttccaagttcttccgtggccctttttgcttattatatcaatccttggtgggagatagaggaagcatttttagtgctattttacaactgaggaaatagaggtttgaagagaactcaggaactctcagggttacccagcattgtgagtgacagagcctggatctgaacgctgatcccataatgcatcctcaagtggtcatcttaagcctcatcctacatctggcagattctgtagctggttctgtaaaggttggtggagaggcaggtccatctgtcacactaccctgccactacagtggagctgtcacatccatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaatgacgactgttccaacgacaactgttccaacaacaatgagcattccaacgacaacgactgttctgacgacaatgactgtttcaacgacaacgagcgttccaacgacaacgagcattccaacaacaacaagtgttccagtgacaacaactgtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaagacccagtggtgctctttgagagtttacgcccatgagtgcagaagactgaacagacatcagcacatcagacgtcttttagaccccaagacaatttttctgtttcagtttcatctggcattccaacatgtcagtgatactgggtagagtaactctctcactccaaactgtgtatagtcaacctcatcattaatgtagtcctaattttttatgctaaaactggctcaatccttctgatcattgcagttttctctcaaatatgaacactttataattgtatgttctttttagaccccataaatcctgtatacatcaaagagaa

SEQ ID NO: 3 - protein, 359 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPTTTVPTTMSIPTTTTVPTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 4 - DNA, 735 bp, Homo sapiens
atgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttag

SEQ ID NO: 5 - DNA, 2926 bp, Homo sapiens
acatctgcttcctgtaggccctctgggcagaagcatgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttagcaaccagaggcatcttctggaagatacacttttgtctttgctattatagatgaatatataagcagctgtactctccatcagtgctgcgtgtgtgtgtgtgtgtgtatgtgtgtgtgtgttcagttgagtgaataaatgtcatcctcttctccatcttcatttccttggccttttcgttctattccattttgcattatggcaggcctagggtgagtaacgtggatcttgatcataaatgcaaaattaaaaaatatcttgacctggttttaaatctggcagtttgagcagatcctatgtctctgagagacacattcctcataatggccagcattttgggctacaaggttttgtggttgatgatgaggatggcatgactgcagagccatcctcatctcattttttcacgtcattttcagtaactttcactcattcaaaggcaggttataagtaagtcctggtagcagcctctatggggagatttgagagtgactaaatcttggtatctgccctcaagaacttacagttaaatggggagacaatgttgtcatgaaaaggtattatagtaaggagagaaggagacatacacaggccttcaggaagagacgacagtttggggtgaggtagttggcataggcttatctgtgatgaagtggcctgggagcaccaaggggatgttgaggctagtctgggaggagcaggagttttgtctagggaacttgtaggaaattcttggagctgaaagtcccacaaagaaggccctggcaccaagggagtcagcaaacttcagattttattctctgggcaggcatttcaagtttccttttgctgtgacatactcatccattagacagcctgatacaggcctgtagcctcttccggccgtgtgtgctggggaagccccaggaaacgcacatgcccacacagggagccaagtcgtagcatttgggccttgatctaccttttctgcatcaatacactcttgagcctttgaaaaaagaacgtttcccactaaaaagaaaatgtggatttttaaaatagggactcttcctaggggaaaaaggggggctgggagtgatagagggtttaaaaaataaacaccttcaaactaacttcttcgaacccttttattcactccctgacgactttgtgctggggttggggtaactgaaccgcttatttctgtttaattgcattcaggctggatcttagaagacttttatccttccaccatctctctcagaggaatgagcggggaggttggatttactggtgactgattttctttcatgggccaaggaactgaaagagaatgtgaagcaaggttgtgtcttgcgcatggttaaaaataaagcattgtcctgcttcctaagacttagactggggttgacaattgttttagcaacaagacaattcaactatttctcctaggatttttattattattattttttcacttttctaccaaatgggttacataggaagaatgaactgaaatctgtccagagctccaagtcctttggaagaaagattagatgaacgtaaaaatgttgttgtttgctgtggcagtttacagcatttttcttgcaaaattagtgcaaatctgttggaaatagaacacaattcacaaattggaagtgaactaaaatgtaatgacgaaaagggagtagtgttttgatttggaggaggtgtatattcggcagaggttggactgagagttgggtgttatttaacataattatggtaattgggaaacatttataaacactattgggatggtgataaaatacaaaagggcctatagatgttagaaatgggtcaggttactgaaatgggattcaatttgaaaaaaatttttttaaatagaactcactgaactagattctcctctgagaaccagagaagaccatttcatagttggattcctggagacatgcgctatccaccacgtagccactttccacatgtggccatcaaccacttaagatggggttagtttaaatcaagatgtgctgttataattggtataagcataaaatcacactagattctggagatttaatatgaataataagaatactatttcagtagttttggtatattgtgtgtcaaaaatgataatattttggatgtattgggtgaaataaaatattaacatta

SEQ ID NO: 6 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 7 - protein, 223 aa, Homo sapiens [sequence omitted]

SEQ ID NO: 8 - DNA, 509 bp, Artificial Sequence
  Description of Artificial Sequence Synthetic polynucleotide
tgggtcctgtcccaggtgcagctgcaggagtcgggcccaggactggtgaagccttcggagaccctgtccctcacctgcactgtctctggtggctccgtcagcagtggtggttactactggagctggatccggcagcccccagggaagggactggagtggattgggtttatctattacactgggagcaccaactacaacccctccctcaagagtcgagtctccatatcagtagacacgtccaagaaccagttctccctgaagctgagctctgtgaccgctgcggacgcggccgtgtattactgtgcgagagattatgactggagcttccactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctct

SEQ ID NO: 9 - protein, 166 aa, Artificial Sequence
  Description of Artificial Sequence Synthetic polypeptide
WVLSQWQLQESGPGLVKPSETLSLTCTVSGGSVSSGGYYWSWIRQPPGKGLEWIGFIYYTGSTNYNPSLKSRWSISWDTSKNQFSLKLSSWTAADAAWYYCARDYDWSFHFDYWGQGTLWTWSSASTKGPSVFPLAPCSRSSESTAAGCVKDYFPEPWTWSWNSGA

SEQ ID NO: 10 - DNA, 503 bp, Artificial Sequence
  Description of Artificial Sequence Synthetic polynucleotide
cagctcctggggctcctgctgctctggttcccaggtgccaggtgtgacatccagatgacccagtctccatcctccctgtctgcatctataggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttaccctctcactttcggcggagggaccaaggtggagatcaaacgaactgtggctgcaccatctgtctcatcttcccgccatctgatgagcagttgaaatctggaactgcctctgttgtgtgcctgctgaataacttctatcccagagaggccaaagtacagtggaaggtggataacgcc

SEQ ID NO: 11 - protein, 168 aa, Artificial Sequence
  Description of Artificial Sequence Synthetic polypeptide
QLLGLLLLWFPGARCDIQMTQSPSSLSASIGDRWTITCRASQGIRNDLGWYQQKPGKAPKRLIYAASSLQSGVPSRFSGSGSGTEFTLTISSLQPEDFATYYCLQHNSYPLTFGGGTKVEIKRTVAAPSVFIFPPSDEQLKSGTASVVCLLNNFYPREAKVQWKVDNA

SEQ ID NO: 12 - DNA, 468 bp, Artificial Sequence
  Description of Artificial Sequence Synthetic polynucleotide
cagtgtgaggtgcagctggtggagtctgggggaggcttggtccagcctggggggtccctgagactctcctgtgcagcctctggattcacctttactaactattggatgagctgggtccgccaggctccagggaaggggctggagtgggtggccaacatacagcaagatggaagtgagaaatactatgtggactctgtgaggggccgattcaccatctccagagacaacgccaagaactcactgtatctgcaaatgaacagcctgagagccgaggactcggctgtgtattactgtgcgagatgggactactggggccagggaaccctggtcaccgtccctcagcctccaccaagggcccatcggtcttccccctggcgccctgctccaggagcacctccgagagcacagcggccctgggctgcctggtcaaggactacttccccgaaccggtgagcggtgtcgtggaac

SEQ ID NO: 13 - protein, 156 aa, Artificial Sequence
  Description of Artificial Sequence Synthetic polypeptide
QCEVQLVESGGGLVQPGGSLRLSCAASGFTFTNYWMSWVRQAPGKGLEWVANIQQDGSEKYYVDSVRGRFTISRDNAKNSLYLQMNSLRAEDSAVYYCARWDYWGQGTLVTVSSASTKGPSVFPLAPCSRSTSESTAALGCLVKDYFPEPVSGVVE

SEQ ID NO: 14 - DNA, 453 bp, Artificial Sequence
  Description of Artificial Sequence Synthetic polynucleotide
cttctggggctgctaatgctctgggtccctggatccagtggggatattgtgatgacccagactccactctcctcaactgtcatccttggacagccggcctccatctcctgcaggtctagtcaaagcctcgtacacagtgatggaaacacctacttgaattggcttcagcagaggccaggccagcctccaagactccaatttatatgatttctaaccggttctctggggtcccagacagattcagtggcagtggggcagggacagatttcacactgaaaatcagcagggtggaagctgaggatgtcggggtttattactgcatgcaagctacagaatctcctcagacgttcggccaagggaccaaggtggaaatcaaacgaactgtggctgcaccatctgtcttcatcttcccgccatctgatgagcagttgaaatctggaagggcctctgttg

SEQ ID NO: 15 - protein, 150 aa, Artificial Sequence
  Description of Artificial Sequence Synthetic polypeptide
LLGLLMLWVPGSSGDIVMTQTPLSSTWILGQPASISCRSSQSLVHSDGNTYLNWLQQRPGQPPRLLIYMISNRFSGVPDRFSGSGAGTDFLKISRVEAEDVGVYYCMQATESPQTFGQGTKVEIKRTVAAPSVFIFPPSDEQLKSGRASV

SEQ ID NO: 16 - DNA, 525 bp, Artificial Sequence
  Description of Artificial Sequence Synthetic polynucleotide
gagcagtcggggggaggcgtggtaaagcctggggggtctcttagactctcctggcagcctctggattcactttcagtaacgcctggatgacctgggtccgccaggctccagggaaggggctggagtgggttggccgtattaaaaggagaactgatggtgggacaacagactacgctgcacccgtgaaaggcagattcaccatctcaagagatgattcaaaaaacacgctgtatctgcaaatgaacaacctgaaaaacgaggacacagccgtgtattactgtacctcagtcgataatgacgtggactactggggccagggaaccctggtcaccgtccctcagctccaccaagggcccaccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactct

SEQ ID NO: 17 - protein, 177 aa, Artificial Sequence
  Description of Artificial Sequence Synthetic polypeptide
  MOD_RES (1)..(4): Any amino acid
XXXXEQSGGGWWKPGGSLRLSCAASGFTFSNAWMTWWRQAPGKGLEWVGRIKRRTDGGTTDYAAPVKGRFTISRIDDSKNTLYLQMNNLKNEDTAWYYCTSVDNDVDYWGGGTLWTVSSASTKGPSVFPLAPCSRSTSESAAGCVKDYFPEPWTWSWNSGAISGWHTFPAVLQSSGL

SEQ ID NO: 18 - DNA, 443 bp, Artificial Sequence
  Description of Artificial Sequence Synthetic polynucleotide
ctgactcagtctccactctccctgcccgtcacccctggagagccggcctccatctcctgcaggtctagtcagagcctcctgcatagtaatggatacaactatttggattggtacctgcagaagccagggcagtctccacagctcctgatctattgggttctaatcgggcctccggggtccctgacaggttcagtggcagtggatcaggcacagattttacactgaaaatcagcagagtggaggctgaggatattggtctttattactgcatgcaagctctacaaactccgctcactttcggcggagggaccaaggtggacatcaaacgaactgtggctgcaccatctgtcttcatcttcccgccatctgatgagcagttgaaatctggaactgcctctgttgtggcctgctgaataacttcacccagagaggccaaagtacag

SEQ ID NO: 19 - protein, 149 aa, Artificial Sequence
  Description of Artificial Sequence Synthetic polypeptide
  MOD_RES (1)..(3): Any amino acid
XXXLTQSPLSLPWTPGEPASISCRSSQSLLHSNGYNYLDWYLQKPGQLLIYLGSNRASGVPDRFSGSGSGTDFTLKISRWEAEDIGLYYCMQALQTPLTFGGGTKWDIKRTVAAPSVFIFPPSDEQLKSGTASVVCLLNNFYPREAKVQ

SEQ ID NO: 20 - protein, 5 aa, Artificial Sequence
  Description of Artificial Sequence Synthetic peptide
TYGMS

SEQ ID NO: 21 - protein, 16 aa, Artificial Sequence
  Description of Artificial Sequence Synthetic peptide
TISGGGRDTYYPDTVK

SEQ ID NO: 22 - protein, 11 aa, Artificial Sequence
  Description of Artificial Sequence Synthetic peptide
QDYGNYVWFAY

SEQ ID NO: 23 - protein, 15 aa, Artificial Sequence
  Description of Artificial Sequence Synthetic peptide
RASESVDSYGISFMH

SEQ ID NO: 24 - protein, 7 aa, Artificial Sequence
  Description of Artificial Sequence Synthetic peptide
STSNRGS

SEQ ID NO: 25 - protein, 9 aa, Artificial Sequence
  Description of Artificial Sequence Synthetic peptide
QQSQEVPWT

SEQ ID NO: 26 - protein, 15 aa, Artificial Sequence
  Description of Artificial Sequence Synthetic peptide
RASKGVSTSGYSYLH

SEQ ID NO: 27 - protein, 7 aa, Artificial Sequence
  Description of Artificial Sequence Synthetic peptide
LASYLES

SEQ ID NO: 28 - protein, 9 aa, Artificial Sequence
  Description of Artificial Sequence Synthetic peptide
QHSRDLPLT

SEQ ID NO: 29 - protein, 5 aa, Artificial Sequence
  Description of Artificial Sequence Synthetic peptide
NYYMY

SEQ ID NO: 30 - protein, 17 aa, Artificial Sequence
  Description of Artificial Sequence Synthetic peptide
GINPSNGGTNFNEKFKN

SEQ ID NO: 31 - protein, 11 aa, Artificial Sequence
  Description of Artificial Sequence Synthetic peptide
RDYRFDMGFDY

SEQ ID NO: 32 - protein, 111 aa, Artificial Sequence [sequence omitted]

SEQ ID NO: 33 - protein, 218 aa, Artificial Sequence
  Description of Artificial Sequence Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 120 aa, Artificial Sequence [sequence omitted]

SEQ ID NO: 35 - protein, 447 aa, Artificial Sequence
  Description of Artificial Sequence Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 108 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 37 - protein, 121 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 38 - protein, 5 aa, Artificial Sequence
  Description of Artificial Sequence Synthetic peptide
RYWMS

SEQ ID NO: 39 - protein, 17 aa, Artificial Sequence
  Description of Artificial Sequence Synthetic peptide
NIKQDGSEKYYVDSVKG

SEQ ID NO: 40 - protein, 12 aa, Artificial Sequence
  Description of Artificial Sequence Synthetic peptide
EGGWFGELAFDY

SEQ ID NO: 41 - protein, 12 aa, Artificial Sequence
  Description of Artificial Sequence Synthetic peptide
RASQRVSSSYLA

SEQ ID NO: 42 - protein, 7 aa, Artificial Sequence
  Description of Artificial Sequence Synthetic peptide
DASSRAT

SEQ ID NO: 43 - protein, 9 aa, Artificial Sequence
  Description of Artificial Sequence Synthetic peptide
QQYGSLPWT

SEQ ID NO: 44 - protein, 40 aa, Artificial Sequence
  Description of Artificial Sequence Synthetic polypeptide
  MISC_FEATURE (1)..(40): This sequence may encompass 1-8 “Gly Gly Gly Gly Ser” repeating units
GGGGSGGGGSGGGGSGGGGSGGGGSGGGGSGGGGSGGGGS

(19) United States
(12) Patent Application Publication
(10) Pub. No.: US 2022/0144950 A1
(43) Pub. Date: May 12, 2022

(54) TARGETING REGULATORY B CELLS AND THEIR REGULATORS FOR CANCER IMMUNOTHERAPY

(71) Applicant: THE BRIGHAM AND WOMEN'S HOSPITAL, INC., Boston, MA (US)

(72) Inventors: Vijay K. KUCHROO, Newton, MA (US); Sheng XIAO, Newton, MA (US); Lloyd BOD, Brookline, MA (US)

(73) Assignee: THE BRIGHAM AND WOMEN'S HOSPITAL, INC., Boston, MA (US)

(21) Appl. No.: 17/438,545

(22) PCT Filed: Mar. 13, 2020
(86) PCT No.: PCT/US2020/022588

§ 371 Date: Sep. 13, 2021

Related U.S. Application Data
(60) Provisional application No. 62/817,856, filed on Mar. 13, 2019.

(51) Int. Cl. C07K 16/28; C12N 15/113; C12N 5/0781; A61P 35/00

(52) U.S. Cl. CPC C07K 16/2818; C12N 15/113; A61K 2039/505; A61P 35/00; C07K 2317/76; C12N 5/0635

(57) ABSTRACT

Provided herein are methods and compositions related to the targeting of Bregs for the treatment of diseases and disorders involving inappropriate suppression of B cell-mediated immune function.

### CROSS-REFERENCE TO RELATED APPLICATIONS

This application is an International Application which designated the U.S., and which claims the benefit under 35 U.S.C. § 119(e) of U.S. Provisional Application No. 62/817,856 filed on Mar. 13, 2019, the content of which is incorporated herein by reference in its entirety.

### GOVERNMENT SUPPORT

This invention was made with government support under Grant Nos. 5P01AI073748-08 and 1P01AI129880-01A1 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 12, 2020, is named 043214-092490WOPT_SL.txt and is 45,918 bytes in size.

### TECHNICAL FIELD

The technology described herein relates to immunotherapy.

### BACKGROUND

The immune system is a collection of organs, cells and specialized tissues that work together to defend the body against foreign invaders and diseased cells. A healthy immune system can recognize foreign or aberrant cells and target them for destruction. However, chronic immune disorders, particularly those associated with aberrant immune tolerance mechanisms, such as chronic infection and cancer, can wreak havoc. According to the most recent data from the World Health Organization, ten million people around the world were diagnosed with cancer in 2000, and six million died from it. Moreover, statistics indicate that the cancer incidence rate is on the rise around the globe. In America, for example, projections suggest that fifty percent of those alive today will be diagnosed with some form of cancer at some point in their lives.

The dynamic relationship between the immune system and cancer development has been well described over the past few decades. The tumor cell microenvironment plays a crucial role in modulating immune responses during cancer progression. For survival, tumors have developed numerous immunosuppressive mechanisms to promote their own growth and to successfully evade the host immune system. Current immunotherapeutic strategies focus extensively upon the augmentation of T-cell-mediated immunity. However, over the past decade, a population of suppressor B cells, collectively known as regulatory B cells (Bregs), have been associated with the inhibition of excessive inflammation and a considerable body of evidence has demonstrated the significance of Bregs cells in diverse models of autoimmunity, infection, and cancer. However, a lack of reliable exclusive cell surface markers identifying Breg cells and lack of fully understanding their regulatory mechanisms have hindered the understanding of Breg biology and their clinical usage.

### SUMMARY

The compositions and methods described herein are based, in part, on the discovery that regulatory B cells (Bregs) differentially express a specific set of cell-surface markers, including TIM-1 (T-Cell Ig and mucin domain protein 1). Bregs are known to restrain inflammation. Defects in Breg number and function have been shown to promote autoimmune disease, while increased number and function of Bregs promote tumor growth. As shown herein, TIM-1, a transmembrane glycoprotein that is expressed on Bregs, is functionally required for Breg suppressive function. Loss of Tim-1 expression in B cells impaired Breg function. Hosts with TIM-1 deficiency specifically in B-cells not only developed more severe induced autoimmune diseases but also with age displayed spontaneous multiorgan tissue inflammation, thus indicating that TIM-1 expression in Bregs is critical for their immunosuppressive function. Tim-1+ Bregs differentially express a set of cohinibitory molecules, many of which are regulated by Tim-1 expression and signaling. Of the negative effector molecules, cohinhibitory molecules such as TIGIT (T-Cell Immunoreceptor with Ig and ITIM Domans) are also required for Breg function. Hosts with TIGIT deficiency specifically in B-cells not only developed spontaneous inflammatory disease, but also showed more severe autoimmune inflammation upon induction.

Hosts with B-cell specific deletion of either TIM-1 or TIGIT strongly inhibit tumor development.

Accordingly, as described herein, compositions and methods targeting B cell expression of TIM-1 or coinhibitory molecules, such as TIGIT can be used to provide novel therapeutic strategies for modulating immune suppression and treating diseases mediated or impacted by immune suppression mechanisms, such as autoimmune diseases, chronic infection and cancer.

In one aspect, described herein is a method of treating a disease or disorder involving inappropriate immunosuppression, the method comprising administering to a subject in need thereof, a therapeutically effective amount of an inhibitor of TIM-1 expression or activity in B cells, thereby treating the disease or disorder.

In one embodiment of any of the aspects, the disease or disorder is selected from cancer and chronic infection.

In another embodiment of any of the aspects, the inhibitor of TIM-1 is targeted to B cells.

In another embodiment of any of the aspects, the B cells comprise Regulatory B cells (Bregs).

In another embodiment of any of the aspects, the inhibitor of TIM-1 comprises a TIM-1 inhibitory moiety and a B cell targeting moiety.

In another embodiment of any of the aspects, the TIM-1 inhibitory moiety is selected from the group consisting of an antibody or antigen-binding fragment thereof, a small molecule, a peptide or polypeptide, a nucleic acid, and a therapeutic virus.

In another embodiment of any of the aspects, the TIM-1 inhibitory moiety comprises an antibody or antigen-binding domain thereof that specifically binds TIM-1.

In another embodiment of any of the aspects, the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.

In another embodiment of any of the aspects, the B cell targeting moiety comprises a moiety that specifically binds to a B cell-specific cell-surface polypeptide.

In another embodiment of any of the aspects, the B cell-specific cell surface polypeptide is selected from the group consisting of CD19, CD20, and CD22.

In another embodiment of any of the aspects, the B cell targeting moiety comprises an antibody or antigen-binding fragment thereof, an aptamer, or a natural ligand that specifically binds the B cell-specific cell surface polypeptide.

In another embodiment of any of the aspects, the inhibitor of TIM-1 reduces expression or activity of one or more selected from the group of TIGIT, TIM-3, LAG3, CTLA4, PD-1, CD39, and CD73 in B cells.

In another embodiment of any of the aspects, further comprising administering a therapeutically effective amount of an inhibitor of the expression or activity of one or more immune checkpoint polypeptides.

In another embodiment of any of the aspects, the one or more immune checkpoint polypeptides are selected from the group consisting of TIGIT, TIM-3, LAG3, CTLA4, and PD-1.

In another embodiment of any of the aspects, further comprising, administering an inhibitor of TIGIT expression or activity.

In another embodiment of any of the aspects, the inhibitor of TIGIT expression or activity is targeted to B cells.

In another embodiment of any of the aspects, the inhibitor of TIGIT expression or activity is selected from the group consisting of an antibody or antigen-binding fragment thereof, a small molecule, a peptide or polypeptide, a nucleic acid and a therapeutic virus.

In another embodiment of any of the aspects, the antibody or antigen-binding fragment thereof comprises the CDRs of a TIGIT-specific antibody in Table 4.

In another embodiment of any of the aspects, the nucleic acid is selected from the group consisting of an RNA interference (RNAi) molecule, a short interfering RNA (siRNA), a short hairpin RNA (shRNA), a micro-RNA (miRNA), an aptamer, and a CRISPR-Cas system.

In another embodiment of any of the aspects, the inhibitor of TIM1 and the inhibitor of TIGIT expression or activity are comprised by a multispecific inhibitory agent comprising an inhibitor of TIM-1 expression or activity and an inhibitor of TIGIT expression or activity.

In another embodiment of any of the aspects, the multispecific inhibitory agent comprises one or more antibody antigen-binding domains.

In another aspect, described herein is a method of reducing B cell-mediated immunosuppression in a subject in need thereof, the method comprising administering to a subject in need thereof, a therapeutically effective amount of an inhibitor of TIM-1 expression or activity in B cells, thereby reducing B cell mediated immunosuppression in the subject.

In one embodiment of any of the aspects, the inhibitor of TIM-1 is targeted to B cells.

In another embodiment of any of the aspects, the B cells comprise Regulatory B cells (Bregs).

In another embodiment of any of the aspects, the inhibitor of TIM-1 comprises a TIM-1 inhibitory moiety and a B cell targeting moiety.

In another embodiment of any of the aspects, the TIM-1 inhibitory moiety is selected from the group consisting of an antibody or antigen-binding fragment thereof, a small molecule, a peptide or polypeptide, a nucleic acid and a therapeutic virus.

In another embodiment of any of the aspects, the TIM-1 inhibitory moiety comprises an antibody or antigen-binding domain thereof that specifically binds TIM-1.

In another embodiment of any of the aspects, the nucleic acid is selected from the group consisting of an RNA interference (RNAi) molecule, a short interfering RNA (siRNA), a short hairpin RNA (shRNA), a micro-RNA (miRNA), an aptamer, and a CRISPR-Cas system.

In another embodiment of any of the aspects, the B cell targeting moiety comprises a moiety that specifically binds to a B cell-specific cell-surface polypeptide.

In another embodiment of any of the aspects, the B cell-specific cell surface polypeptide is selected from the group consisting of CD19, CD20 and CD22.

In another embodiment of any of the aspects, the B cell targeting moiety comprises an antibody or antigen-binding fragment thereof, or an aptamer.

In another embodiment of any of the aspects, the inhibitor of TIM-1 reduces expression or activity of one or more of TIGIT, TIM-3 LAG3, CTLA4 or PD-1 in Breg cells.

In another embodiment of any of the aspects, further comprising administering a therapeutically effective amount of an inhibitor of the expression or activity of one or more immune checkpoint polypeptides.

In another embodiment of any of the aspects, the one or more immune checkpoint polypeptides are selected from the group consisting of TIGIT, TIM-3, LAG3, CTLA4, PD-1, CD39, and CD73.

In another embodiment of any of the aspects, further comprising administering an inhibitor of TIGIT expression or activity.

In another embodiment of any of the aspects, the inhibitor of TIGIT expression or activity is selected from the group consisting of an antibody or antigen-binding fragment thereof, a small molecule, a peptide or polypeptide, a nucleic acid and a therapeutic virus.

In another embodiment of any of the aspects, the antibody or antigen-binding fragment thereof comprises the CDRs of TIGIT-specific antibody in Table 4.

In another embodiment of any of the aspects, the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.

In another embodiment of any of the aspects, the inhibitor of TIM1 and the inhibitor of TIGIT expression or activity are comprised by a multi specific inhibitory agent comprising an inhibitor of TIM-1 expression or activity and an inhibitor of TIGIT expression or activity.

In another embodiment of any of the aspects, the multispecific inhibitory agent comprises one or more antibody antigen-binding domains.

In another aspect, described herein is a composition comprising an inhibitor of TIM-1 expression or activity that is targeted to B cells.

In one embodiment of any of the aspects, the inhibitor of TIM-1 comprises a moiety that inhibits the expression or activity of TIM-1 and a moiety that specifically binds a B cell-specific cell surface polypeptide.

In another embodiment of any of the aspects, the moiety that inhibits the expression or activity of TIM-1 is selected from the group consisting of an antibody or antigen-binding fragment thereof, a small molecule, a peptide or polypeptide, a nucleic acid and a therapeutic virus.

In another embodiment of any of the aspects, the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.

In another embodiment of any of the aspects, the B cell-specific cell surface polypeptide is selected from the group consisting of CD19, CD20, and CD22.

In another embodiment of any of the aspects, the moiety that specifically binds a B cell-specific cell surface polypeptide comprises an antibody or antigen-binding fragment thereof, or an aptamer.

In another embodiment of any of the aspects, further comprising one or more inhibitors of the expression or activity of one or more immune checkpoint polypeptides.

In another embodiment of any of the aspects, the one or more immune checkpoint polypeptides comprises TIGIT.

In another embodiment of any of the aspects, the inhibitor of TIGIT expression or activity is selected from the group consisting of an antibody or antigen-binding fragment thereof, a small molecule, a peptide or polypeptide, a nucleic acid and a therapeutic virus.

In another embodiment of any of the aspects, the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.

In another embodiment of any of the aspects, the antibody or antigen-binding fragment thereof comprises the CDRs of TIGIT-specific antibody in Table 4.

In another aspect, described herein is a composition comprising an inhibitor of TIGIT expression or activity that is targeted to B cells.

In one embodiment of any of the aspects, the inhibitor of TIGIT expression or activity is selected from the group consisting of an antibody or antigen-binding fragment thereof, a small molecule, a peptide or polypeptide, a nucleic acid and a therapeutic virus.

In another embodiment of any of the aspects, the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.

In another embodiment of any of the aspects, the antibody or antigen-binding fragment thereof comprises the CDRs of a TIGIT-specific antibody in Table 4.

In another aspect, described herein is a method of treating a disease or disorder involving inappropriate immunosuppression, the method comprising administering to a subject in need thereof, a therapeutically effective amount of an inhibitor of TIM-1 expression or activity and a therapeutically effective amount of an inhibitor of PD-1 expression or activity, thereby treating the disease or disorder.

In one embodiment of any of the aspects, the disease or disorder is selected from cancer and chronic infection.

In another embodiment of any of the aspects, the administering comprises administering a multispecific inhibitory agent comprising a TIM-1 inhibitory moiety and a PD-1 inhibitory moiety.

In another embodiment of any of the aspects, the inhibitor of TIM-1 is targeted to B cells.

In another embodiment of any of the aspects, the B cells comprise Regulatory B cells (Bregs).

In another embodiment of any of the aspects, the inhibitor of TIM-1 comprises a TIM-1 inhibitory moiety and a B cell targeting moiety.

In another embodiment of any of the aspects, the TIM-1 inhibitory moiety is selected from the group consisting of an antibody or antigen-binding fragment thereof, a small molecule, a peptide or polypeptide, a nucleic acid, and a therapeutic virus.

In another embodiment of any of the aspects, the TIM-1 inhibitory moiety comprises an antibody or antigen-binding domain thereof that specifically binds TIM-1.

In another embodiment of any of the aspects, the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.

In another embodiment of any of the aspects, the B cell targeting moiety comprises a moiety that specifically binds to a B cell-specific cell-surface polypeptide.

In another embodiment of any of the aspects, the B cell-specific cell surface polypeptide is selected from the group consisting of CD19, CD20, and CD22.

In another embodiment of any of the aspects, the B cell targeting moiety comprises an antibody or antigen-binding fragment thereof, an aptamer, or a natural ligand that specifically binds the B cell-specific cell surface polypeptide.

In another embodiment of any of the aspects, the inhibitor of TIM-1 reduces expression or activity of one or more selected from the group of TIGIT, TIM-3, LAG3, CTLA4, PD-1, CD39, and CD73 in B cells.

In another aspect, described herein is an inhibitor of TIM-1 expression or activity that is targeted to B cells, for use in the treatment of a disease or disorder involving inappropriate immunosuppression.

In one embodiment of any of the aspects, the disease or disorder is cancer or chronic infection.

In another embodiment of any of the aspects, the inhibitor of TIM-1 comprises a moiety that inhibits the expression or activity of TIM-1 and a moiety that specifically binds a B cell-specific cell surface polypeptide.

In another embodiment of any of the aspects, the moiety that inhibits the expression or activity of TIM-1 is selected from the group consisting of an antibody or antigen-binding fragment thereof, a small molecule, a peptide or polypeptide, a nucleic acid and a therapeutic virus.

In another embodiment of any of the aspects, the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.

In another embodiment of any of the aspects, the B cell-specific cell surface polypeptide is selected from the group consisting of CD19, CD20, and CD22.

In another embodiment of any of the aspects, the moiety that specifically binds a B cell-specific cell surface polypeptide comprises an antibody or antigen-binding fragment thereof, or an aptamer.

In another aspect, described herein is a composition comprising an inhibitor of TIM-1 expression or activity and an inhibitor of an immune checkpoint inhibitor polypeptide for use in the treatment of a disease or disorder involving inappropriate immunosuppression.

In one embodiment of any of the aspects, the disease or disorder is cancer or chronic infection.

In another embodiment of any of the aspects, the inhibitor of TIM-1 comprises a moiety that inhibits the expression or activity of TIM-1 and a moiety that specifically binds a B cell-specific cell surface polypeptide.

In another embodiment of any of the aspects, the moiety that inhibits the expression or activity of TIM-1 is selected from the group consisting of an antibody or antigen-binding fragment thereof, a small molecule, a peptide or polypeptide, a nucleic acid and a therapeutic virus.

In another embodiment of any of the aspects, the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.

In another embodiment of any of the aspects, the B cell-specific cell surface polypeptide is selected from the group consisting of CD19, CD20, and CD22.

In another embodiment of any of the aspects, the moiety that specifically binds a B cell-specific cell surface polypeptide comprises an antibody or antigen-binding fragment thereof, or an aptamer.

In another embodiment of any of the aspects, the one or more immune checkpoint polypeptides comprises TIGIT.

In another embodiment of any of the aspects, the inhibitor of TIGIT expression or activity is selected from the group consisting of an antibody or antigen-binding fragment thereof, a small molecule, a peptide or polypeptide, a nucleic acid and a therapeutic virus.

In another embodiment of any of the aspects, the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.

In another embodiment of any of the aspects, the antibody or antigen-binding fragment thereof comprises the CDRs of a TIGIT-specific antibody in Table 4.

In another aspect, described herein is a method of treating a disease or disorder involving inappropriate immunosuppression, the method comprising administering to a subject in need thereof, a therapeutically effective amount of an inhibitor of TIM-1 expression or activity and a therapeutically effective amount of an inhibitor of PD-L1 expression or activity, thereby treating the disease or disorder.

In one embodiment of any of the aspects, the administering comprises administering a multispecific inhibitory agent comprising a TIM-1 inhibitory moiety and a PD-L1 inhibitory moiety.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “inhibitor of TIM-1 expression or activity in B cells” refers to an agent that reduces TIM-1 expression level or activity in B cells by at least 20% when compared to TIM-1 expression level or activity in B cells in the absence of the inhibitor and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. An inhibitor of TIM-1 expression or activity in B cells is targeted to and preferentially inhibits TIM-1 in B cells. That is, such an inhibitor inhibits TIM-1 activity in B cells at least 2× more strongly than in other cells expressing TIM-1 activity. In certain embodiments, such an inhibitor inhibits TIM-1 activity in B cells at least 5×, 10×, 20×, 50×, 100× or more strongly than in other cells expressing TIM-1 activity. In one embodiment, such an inhibitor does not substantially inhibit TIM-1 in non-B cells in vivo.

By “reducing TIM-1 expression” in this context is meant at least a 20%, at least a 25%, at least a 30%, at least a 35%, at least a 40%, at least a 45%, at least a 50%, at least a 55%, at least a 60%, at least a 70%, at least a 75%, at least a 80%, at least a 85%, at least a 90%, at least a 95%, at least a 98% or at least a 99% or greater reduction in the rate or level of expression, in the presence of a given inhibitor as compared to the expression in the absence of the inhibitor. In this context, expression can be measured at the level of mRNA encoding TIM-1 or at the level of TIM-1 protein. To be clear, however, an inhibitor of TIM-1 expression will reduce the level of TIM-1 protein.

As used herein, the term “inhibitor of TIM-1” or “TIM-1 inhibitor” refers to an agent that can reduce and/or inhibit TIM-1 expression or activity. In one embodiment, a TIM-1 inhibitor binds to TIM-1 polypeptide and inhibits TIM-1 activity, or interferes with translation of mRNA encoding TIM-1. Exemplary agents include, but are not limited to an antibody, or antigen-binding fragment thereof, a small molecule, a peptide, polypeptide, nucleic acid, an RNAi interference (RNAi) molecule (including but not limited to a short interfering RNA (siRNA), a short hairpin RNA (shRNA) or a micro-RNA (miRNA)), or an aptamer. In some embodiments, inhibition can be effective at the transcriptional level, for example by reducing or inhibiting mRNA transcription of TIM-1, for example, human TIM-1 (NCBI Gene ID No. 26762). As used herein, “reduction” or “inhibition” does not encompass a complete inhibition or reduction as compared to a reference level. “Complete inhibition” refers to detect a 100% inhibition (i.e. expression or activity that is below detectable limits using a standard assay measuring TIM-1 expression in B cells). TIM-1 expression level can be measured by any means known in the art, including, but not limited to Western blotting, e.g., of sort-purified B cells treated with the inhibitor compared to B cells in the absence of the inhibitor. TIM-1 activity level can be measured by any means known in the art, including, but not limited to, e.g., measuring the IL-10 production in culture supernatants of sort-purified B cells treated with an inhibitor, as compared to B cells in the absence of the inhibitor using, for example a cytokine bead array (CBA) or ELISA. TIM-1 promotes expression of IL-10, which is itself an inhibitor of cytokine expression. Thus, inhibition of TIM-1 whether at the RNA or protein level, leads to a decrease in IL-10 level.

By “reducing TIM-1 activity” in this context is meant at least a at least a 20%, at least a 25%, at least a 30%, at least a 35%, at least a 40%, at least a 45%, at least a 50%, at least a 55%, at least a 60%, at least a 70%, at least a 75%, at least a 80%, at least a 85%, at least a 90%, at least a 95%, at least a 98% or at least a 99% or greater reduction in activity compared to the activity of TIM-1 in B cells in the absence of a TIM-1 inhibitor. In this context, TIM-1 activity level can be measured by measuring IL-10 production in culture supernatants of sort-purified B cells treated with the inhibitor, as compared to B cells in the absence of the inhibitor using, for example, a cytokine bead array (CBA) or ELISA.

As used herein, a “regulatory B cell (Breg)” refers to a subset of B cells or a population thereof that exert immune regulatory functions. Bregs exert inhibitory immune regulatory functions through the production of interleukin (IL)-10 and transforming growth factor-β (TGF-β), among others.

As used herein, a “regulator of B cell-mediated immunosuppression” refers to a molecule, such as TIM-1 and/or TIGIT, the expression or activity of which in Bregs is required for their steady-state or induced regulatory activities, including inhibition of activated T cells, production of IL-10, maintenance of regulatory Foxp3+ Tregs, etc. As used herein, an “inhibitory agent” or “inhibitor,” is an agent that reduces the expression level and/or activity of a target as compared to the expression level and/or activity in the absence of the agent, for example by a statistically significant amount. For the avoidance of doubt, reduction by at least 10%, at least 20%, at least 30%, at least 40%, at least 50%, at least 60%, at least 70%, at least 80%, at least 90%, at least 95% or more can be considered inhibitory. The expression level of a target can be the expression level of a polypeptide target, the expression level of an isoform or variant of a polypeptide target, or the expression level of an RNA transcript encoding the polypeptide target. The activity of a polypeptide target can include, but is not limited to, the ability of the target to bind a normal ligand, the ability of the target to interact with other polypeptides (e.g. downstream signaling partners), and/or the ability of the target polypeptide to effect a downstream response (e.g. phosphorylation levels or gene expression). In some aspects, such inhibitors include inhibitors of TIM-1 and/or TIGIT expression or activity in B cells.

As used herein, a “cell-surface marker” refers to any molecule that is expressed on the surface of a cell. Cell-surface expression usually requires that a molecule possesses a transmembrane domain. Many naturally occurring cell-surface markers are characterized among the “CD” or “cluster of differentiation” molecules. Cell-surface markers vary depending upon cell type, and often provide antigenic determinants to which antibodies can bind, e.g., for targeting of an agent to a desired cell or tissue.

As used herein, the term “B cell-specific” refers to a marker or a phenotype that is characteristic of B cells. Examples include, but are not limited to CD19, CD20 and CD22. Most B cells express the cell-surface marker CD19, which is a B cell-specific marker as the term is used herein. CD19 is the B lymphocyte surface antigen B4, and a component of the B cell co-receptor. Most mature B cells express CD19 and IgM, which is also a B cell-specific marker as the term is used herein. Subsets of B cells will generally express CD19, but will also express one or more additional markers that alone or in combination are characteristic of the given subsets. For example, immature B cells express CD19, CD20, CD34, CD38 and CD45R, but not IgM. Of these markers, CD20 is also B cell-specific, and encodes a calcium channel protein, but CD34, CD38 and CD45R are commonly expressed in other, non-B cells, such that while they can in combination help to identify or characterize a subset of B cells, they are not “B cell-specific” as the term is used herein. Plasma cells, a subset of B cells, lose CD19 expression, but express CD78. Memory B cells express CD20 and CD40, among other markers, in addition to CD19.

As used herein, an agent “targeted to B cells” will, following administration to an individual, be found in association with B cells to a significantly greater extent than is associated with another cell population or fraction that expresses a given objective for inhibition. As used herein, an agent “targeted to B cells” incudes a moiety that specifically binds a B cell-specific cell-surface marker. An agent that is “targeted to B cells” will preferentially localize to B cells relative to the same agent lacking a moiety that targets it to B cells. In this context, “preferentially localize” means the targeted agent will localize to or be found in association with B cells to an extent at least 5× greater than the same agent lacking the targeting moiety when administered in the same agent concentration. In some embodiments, the localization to B cells can be at least 10× greater, 20× greater, 50× greater, 100× greater or more, relative to localization to B cells by the same agent lacking the targeting moiety.

As used herein, “selectively binds” or “specifically binds” or “specific binding” in reference to the interaction of an antibody, or antibody fragment thereof, or a binding protein described herein, means that the interaction is dependent upon the presence of a particular structure (e.g., an antigenic determinant or epitope or target) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. In certain embodiments, a binding protein or antibody or antigen-binding fragment thereof that specifically binds to an antigen binds to that antigen with a K_{D} greater than 10⁻⁶ M, 10⁻⁷ M, 10⁻⁸M, 10⁻⁹ M, 10⁻¹⁰ M, 10⁻¹¹ M, 10⁻¹² M, 10⁻¹³ M, 10⁻¹⁴ M. In other embodiments, a binding protein or antibody or antigen binding fragment thereof that specifically binds to an antigen binds to that antigen with a K_{D} between 10⁻⁶ and 10⁻⁷ M, 10⁻⁶ and 10⁻⁸M, 10⁻⁶ and 10⁻⁹ M, 10⁻⁶ and 10⁻¹⁰ M, 10⁻⁶ and 10⁻¹¹ M, 10⁻⁶ and 10⁻¹² M, 10⁻⁶ and 10⁻¹³M, 10⁻⁶ and 10⁻¹⁴ M, 10⁻⁹ and 10⁻¹⁰ M, 10⁻⁹ and 10⁻¹¹ M, 10⁻⁹ and 10⁻¹² M, 10⁻⁹ and 10⁻¹³ M, 10⁻⁹ and 10⁻¹⁴ M. In some embodiments, a binding protein or antibody or antigen-binding fragment thereof binds to an epitope, with a K_{D} 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay. In certain embodiments, a binding protein or antibody or antigen-binding fragment thereof is said to “specifically bind” an antigen when it preferentially recognizes its target antigen in a complex mixture of proteins and/or macromolecules. Binding proteins, antibodies or antigen-binding fragments that bind to the same or similar epitopes will likely cross-compete (one prevents the binding or modulating effect of the other). Cross-competition, however, can occur even without epitope overlap, e.g., if epitopes are adjacent in three-dimensional space and/or due to steric hindrance.

As used herein, the term “TIM-1 inhibitory moiety” refers to the portion of a targeted TIM-1 inhibitor that inhibits the expression or activity of TIM-1. In some embodiments, the inhibitory moiety is selected from the group consisting of an antibody or antigen-binding fragment thereof, a small molecule, a peptide or a polypeptide, and a nucleic acid. As used herein, the term “B cell targeting moiety” refers to the portion of a targeted TIM-1 inhibitor that specifically binds to a B cell-specific cell-surface polypeptide. In some embodiments, the B cell targeting moiety specifically binds to a B cell-specific cell-surface protein.

As used herein, the term “inhibitor of TIGIT” refers to an agent that can reduce or inhibit TIGIT expression or activity. In one embodiment, and inhibitor of TIGIT binds to a TIGIT polypeptide and inhibits its activity. In another embodiment, a TIGIT inhibitor interferes with translation of mRNA encoding TIGIT. Exemplary agents include, but are not limited to an antibody, or antigen-binding fragment thereof, a small molecule, a peptide, polypeptide, nucleic acid, an RNAi interference (RNAi) molecule, a short interfering RNA (siRNA), a short hairpin RNA (shRNA), a micro-RNA (miRNA), or an aptamer. In some embodiments, inhibition can be effective at the transcriptional level, for example by reducing or inhibiting mRNA transcription of TIGIT, for example, human TIGIT (NCBI Gene ID No. 201633).

It is recognized herein, that TIM-1 modulates the expression of TIGIT, TIM-3, LAG-3, CTLA-4 and PD-1 in B cells. Thus it could be viewed that an inhibitor of TIM-1 activity or expression is also an inhibitor of TIGIT, TIM-3, LAG-3, CTLA-4 or PD-1 activity or expression. However, where the methods described herein indicate treatment with an inhibitor of TIM-1 and an inhibitor of any of TIGIT, TIM-3, LAG-3, CTLA-4 and PD-1, it should be understood that the method refers to treatment with an agent separate from and in addition to an agent that binds to or directly inhibits the expression of TIM-1.

As used herein, the term “inhibitor of TIGIT expression or activity in B cells” refers to an agent that reduces TIGIT expression level or activity in B cells by at least 20% when compared to TIGIT expression level or activity in B cells in the absence of the inhibitor and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. An “inhibitor of TIGIT expression or activity in B cells” is targeted to and preferentially inhibits TIGIT in B cells. That is, such an inhibitor inhibits TIGIT activity in B cells at least 2× more strongly than in other cells expressing TIGIT activity. An “inhibitor of “TIGIT activity or expression in B cells” can include a protein-binding agent that permits inhibition of TIM-1 and/or TIGIT signaling. Such agents include, but are not limited to, antibodies, multi-specific protein-binding agents, protein-binding agents, small molecules, recombinant protein, peptides, aptamers, avimers and protein-binding derivatives, portions or fragments thereof. In some embodiments, antisense oligonucleotides represent another class of agents that are useful in the compositions and methods described herein. This class of agents and methods for preparing and using them are all well-known in the art, as are ribozyme and miRNA molecules. See, e.g., PCT US2007/024067 (which is incorporated by reference herein in its entirety) for a thorough discussion.

An “agent” can be any chemical, entity or moiety, including without limitation synthetic and naturally-occurring proteinaceous and non-proteinaceous entities. In some embodiments, an agent is a nucleic acid, nucleic acid analog, protein, antibody, peptide, aptamer, oligomer of nucleic acids, amino acids, or carbohydrates including without limitation a protein, oligonucleotide, ribozyme, DNAzyme, glycoprotein, siRNAs, lipoprotein and/or a modification or combinations thereof etc. In certain embodiments, agents are small molecule chemical moieties. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

An agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertions and other variants.

In one embodiment, such an inhibitor does not substantially inhibit TIGIT in non-B cells in vivo. As used herein, “reduction” or “inhibition” does not encompass a complete inhibition or reduction as compared to a reference level. “Complete inhibition” refers to detect a 100% inhibition (i.e. expression or activity that is below detectable limits using a standard assay measuring TIGIT expression in B cells). TIGIT expression level can be measured by any means known in the art, including, but not limited to Western blotting, e.g., of sort-purified B cells treated with the inhibitor compared to B cells in the absence of the inhibitor. TIGIT activity level can be measured by any means known in the art, including, but not limited to, e.g., measuring the IL-10 production in culture supernatants of sort-purified B cells treated with an inhibitor, as compared to B cells in the absence of the inhibitor using a cytokine bead array (CBA) or ELISA. TIGIT promotes expression of the inhibitory cytokine IL-10. Thus, inhibition of TIGIT whether at the RNA or protein level, leads to a decrease in IL-10 level.

By “reducing TIGIT expression” in this context is meant at least a 20%, at least a 25%, at least a 30%, at least a 35%, at least a 40%, at least a 45%, at least a 50%, at least a 55%, at least a 60%, at least a 70%, at least a 75%, at least a 80%, at least a 85%, at least a 90%, at least a 95%, at least a 98% or at least a 99% greater reduction in the rate of expression or level, in the presence of a given agent. In this context, expression can be measured at the level of mRNA encoding TIGIT or at the level of TIGIT protein. To be clear, however, an inhibitor of TIGIT expression will reduce the level of TIGIT protein.

By “reducing TIGIT activity” in this context is meant at least a at least a 20%, at least a 25%, at least a 30%, at least a 35%, at least a 40%, at least a 45%, at least a 50%, at least a 55%, at least a 60%, at least a 70%, at least a 75%, at least a 80%, at least a 85%, at least a 90%, at least a 95%, at least a 98% or at least a 99% or even 100% reduction in activity compared to the activity of TIGIT in B cells in the absence of a TIGIT inhibitor. In this context, TIGIT activity level can be measured by measuring the IL-10 production in culture supernatants of sort-purified B cells treated with the inhibitor, as noted above.

In some embodiments of any of the aspects, an agent that inhibits TIM-1 or TIGIT is an inhibitory nucleic acid. Examples include RNAi molecules or antisense molecules. In another embodiment, and inhibitory nucleic acid includes an aptamer that binds and inhibits the activity of a target polypeptide.

Double-stranded RNA molecules (dsRNA) have been shown to block gene expression in a highly conserved and target-specific regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. Various embodiments of inhibitory RNAs include, for example RNAi molecules, shRNA molecules and miRNA molecules. shRNAs and miRNAs can be, for example, expressed from a construct introduced to cells. The use of these iRNAs permits the targeted degradation of mRNA transcripts, resulting in decreased expression and/or activity of the target.

As used herein, “small molecule inhibitors” include, but are not limited to, small peptides or peptide-like molecules, soluble peptides, and non-peptidyl organic or inorganic compounds. A small molecule inhibitor or antagonist can have a molecular weight of about 100 to about 20,000 Daltons (Da), for example about 500 to about 15,000 Da, or about 1000 to about 10,000 Da.

As used herein, “antibodies” or “antigen-binding fragments thereof” include monoclonal, human, humanized or chimeric antibodies, single chain antibodies, Fab fragments, F(ab′) fragments, fragments produced by a Fab expression library, and/or antigen-binding fragments of any of the above. Antibodies can also refer to immunoglobulin molecules and immunologically active portions that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

As used herein, a receptor-binding unit or antibody reagent specifically binds to a target receptor molecule present on the cell-surface, with a K_{D} of 10-5 M (10000 nM) or less, e.g., 10-6 M or less, 10-7 M or less, 10-8 M or less, 10-9 M or less, 10 10 M or less, 10-11 M or less, or 10-12 M or less and binds to that target at least 100×, or 1000×, or 10,000× and preferably more strongly than it binds to another cell-surface receptor. The person of ordinary skill in the art can determine appropriate conditions under which the polypeptide agents described herein selectively bind the targets using any suitable methods, such as titration of a receptor-binding unit or antibody reagent in a suitable cell binding assay.

As used herein, an “antigen-binding fragment” refers that portion of an antibody that is necessary and sufficient for binding to a given antigen. At a minimum, an antigen binding fragment of a conventional antibody will comprise six complementarity determining regions (CDRs) derived from the heavy and light chain polypeptides of an antibody arranged on a scaffold that permits them to selectively binds the antigen. A commonly used antigen-binding fragment includes the V_{H} and V₂ domains of an antibody, which can be joined either via part of the constant domains of the heavy and light chains of an antibody, or, alternatively, by a linker, such as a peptide linker. Non-conventional antibodies, such as camelid and short antibodies have only 2 heavy chain sequences, denoted, for example V_{HH}. These can be used in a manner analogous to V_{H}/VL-containing antigen-binding fragments. Non-limiting examples of antibody fragments encompassed by the term antigen-binding fragment include: (i) a Fab fragment, having V_{L}, C_{L}, V_{H} and C_{H}1 domains; (ii) a Fab′ fragment, which is a Fab fragment having one or more cysteine residues at the C-terminus of the C_{H}1 domain; (iii) an Fd fragment having V_{H} and C_{H}1 domains; (iv) a Fd′ fragment having V_{H} and C_{H}1 domains and one or more cysteine residues at the C-terminus of the C_{H}1 domain; (v) an Fv fragment having the V_{L} and V_{H} domains of a single arm of an antibody; (vi) a dAb fragment (Ward et al., Nature 341, 544-546 (1989)) which consists of a V_{H} domain; (vii) F(ab′)₂ fragments, a bivalent fragment including two Fab′ fragments linked by a disulphide bridge at the hinge region; (viii) single chain antibody molecules (e.g., single chain Fv; scFv) (Bird et al., Science 242:423-426 (1988); and Huston et al., PNAS (USA) 85:5879-5883 (1988)); (ix) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); and (x) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870).

An “isolated antibody” is intended to refer to an antibody that is substantially free of other antibodies having different antigenic specificities (e.g., an isolated antibody that specifically binds to TIM-1 or TIGIT is substantially free of antibodies that specifically bind antigens other than TIM-1 or TIGIT. An isolated antibody that specifically binds to TIM-1, or TIGIT may, however, have cross-reactivity to other antigens, such as to TIM-1 or TIGIT molecules from other species. Moreover, an isolated antibody can be substantially free of other cellular material and/or chemicals.

The term “therapeutically effective amount” refers to an amount of an inhibitor as described herein, that is effective to treat a disease or disorder as the terms “treat” or “treatment” are defined herein. Amounts will vary depending on the specific disease or disorder, its state of progression, age, weight and gender of a subject, among other variables. Thus, it is not possible to specify an exact “effective amount”. However, for any given case, an appropriate “effective amount” can be determined by one of ordinary skill in the art using only routine experimentation.

As used herein, the term “therapeutic virus” refers to a genetically engineered or naturally occurring virus that can be used as a therapeutic agent to treat disease.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total). The term “treatment” of a disease also includes providing relief from the symptoms or side-effects of the disease (including palliative treatment).

The terms “decrease”, “reduce”, “reduction”, or “inhibit” are all used herein to mean a decrease by a statistically significant amount. In some embodiments, “reduce,” “reduction”, “decrease” or “inhibit” means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, “immunosuppression” refers to an active process whereby one or more components of the adaptive or innate immune system is or are prevented from acting against a given target. The immune system includes naturally immunosuppressive function mediated by immune inhibitory receptors or cytokines expressed on the surface of an immune cell, and their interactions with their ligands. For example, cytotoxic CD8 T cells can enter a state of “functional exhaustion,” or “unresponsiveness” whereby they express inhibitory receptors that inhibit or prevent antigen-specific responses, such as proliferation and inflammatory cytokine production. Accordingly, by inhibiting the activity and/or expression of such inhibitory receptors or cytokines, an immune response to a persistent infection or to a cancer or tumor that is suppressed, inhibited, or ineffective, can be enhanced or” un-inhibited”.

As used herein, the term “inappropriate immunosuppression” refers to immunosuppression that inhibits or renders the immune response to a given pathological condition less effective. Examples of inappropriate immunosuppression include, for example, immunosuppression mediated by a tumor or the state in a tumor microenvironment, or immunosuppression-mediated by a pathogenic organism.

As used herein, an “immune response” refers to a response by one or more cells of the immune system to a pathological or pathogenic stimulus. Cells of the immune system include, but are not limited to B cells, regulatory B cells, T cells, regulatory T cells, antigen-presenting cells, dendritic cells, monocytes, macrophages, NKT cells, NK cells, basophils, eosinophils and neutrophils.

As used herein, the term “disease or disorder involving inappropriate immunosuppression” refers to a disease or disorder in which the function of the immune response is below a desired level, e.g. a level that can treat or prevent at least one symptom of the disease or disorder. As a non-limiting example, inappropriate immune suppression can be associated with certain cancer tumors in which cells of the immune system fail to attack or are prevented from attacking the tumor, such that thus the immune system fails to effectively reduce or prevent tumor growth.

As used herein, a “subject” is a human or a non-human animal. Usually the non-human animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of diseases including diseases and disorders involving inappropriate immunosuppression. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

As used herein, a “subject in need” of treatment for a particular condition can be a subject having that condition, diagnosed as having that condition, or at risk of developing that condition.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1A-1I demonstrate the role of TIM-1 and/or TIGIT in B cell-mediated tumor growth. FIG. 1A. MC38 cells were injected subcutaneously (s.c.) at day 0 in 8 to 10-week old control and Tim-1 BKO mice (n=10 mice/group) and tumor growth was measured with a caliper for 17 days. FIG. 1B. At day 17, tumor infiltrating T cells from the MC38-bearing mice were analyzed by flow cytometry. Representative FACS plots gated on tumor infiltrating CD8+ and CD4+ T cells showed PD1+Tim-3+ exhausted cells, IFN-γ-producing CD8+ T cells, and CD4+Foxp3+ Tregs. FIG. 1C. B16F10 cells were injected s.c. at day 0 in 8 to 10-week old control (n=12) and Tim-1 BKO mice (n=14) and tumor growth was measured for 20 days (left panel); At day 20, B16F10 tumor weight was quantitated (right panel). FIG. 1D. B16F10 cells were injected s.c. at day 0 in 8 to 10-week old control (n=10) and Tigit BKO mice (n=10) and tumor growth was measured for 20 days. FIG. 1E. WT mice (n=7-8/group) were injected s.c. with B16F10 cells at day 0, and control rIgG2a or anti-Tim-1 mAb (clone 3B3, 250m) was injected on days 10, 12 and 14. tumor growth was measured for 18 days. Error bars depicts SEM. Statistical analyses were performed using unpaired t-test or Mann-Whitney test. *, p<0.05; **, p<0.01; ***, p<0.001. TIM-1 pharmacological targeting regulates tumour growth and synergize with anti-PD1 immunotherapy. FIG. 1F. CD19CrexTIM-1fl/fl (TIM-1BKO) and CD19Cre (control) mice were implanted with B16F10 melanoma, and treated with anti-TIM-1 Ab (250 μg, clone 3B3) or isotype control on days 7, 9 and 11 and tumor growth was monitored (FIG. 1F). Reduced tumor growth was found upon anti-Tim-1 treatment that was abolished in absence of Tim-1-expressing B cells. This result shows a modulation of Tim-1+ B cells function using anti-Tim-1 immunotherapy. Moreover, the efficacy of combining anti-Tim-1 and anti-PD-1 therapy was evaluated. C57B16/J (WT) mice were implanted with B16F10 melanoma and treated with anti-TIM-1 as well as with three doses of anti-PD1 (200m, clone RMP1-14) antibody or isotype control (FIG. 1G). Combined Tim-1 and PD-1 blockade significantly reduced B16F10 control tumor growth. Thus, these data show that blockade of Tim-1 function enhances antitumor responses to contemporary immune checkpoint blockade and depends on Tim-1+ B cells function.

### DETAILED DESCRIPTION

The compositions and methods described herein are based in part on the discovery that in addition to restraining inflammation in induced-disease settings, regulatory B cells (Bregs) suppress natural anti-tumor immune responses. The compositions and methods described herein are also based, in part, on the discovery of the effects of TIM-1 and/or TIGIT on Breg function. As shown herein, TIM-1 (T-Cell Ig and mucin domain protein 1), a transmembrane glycoprotein that is expressed on Bregs, is functionally required for Breg immunosuppressive function, by regulating optimal expression of coinhibitory molecules such as TIGIT (T-Cell Immunoreceptor with Ig and ITIM Domains). Loss of Tim-1 expression in B cells impairs Breg function. Hosts with TIM-1 deficiency specifically in B-cells not only developed more severe induced autoimmune diseases but also with age displayed spontaneous multiorgan tissue inflammation. Hosts with B-cell specific deletion of either TIM-1 or TIGIT strongly inhibit tumor development.

Accordingly, as described herein, compositions and methods targeting TIM-1 and coinhibitory molecules, such as TIGIT, in B cells can be used to provide therapeutic strategies for modulating immune suppression and treating diseases mediated or impacted by immune suppression mechanisms, such as cancer and chronic infection. The following describes considerations to facilitate the practice of the methods and compositions described herein for modulating immune suppression and treating diseases or disorders associated with such suppression.

Regulators of B Cell Mediated Immunosuppression

The compositions and methods described herein target one or more regulators of B cell-mediated immunosuppression, for example, TIM-1 and/or TIGIT.

TIM-1 (T-Cell Immunoglobulin Mucin Domain-1)

Human TIM-1 is a 359 amino acid polypeptide. TIM-1 polypeptides also include allelic, splice variants, and processed forms thereof that function in promoting Breg immunosuppressive activity.

TIM-1 was originally identified in African green monkeys as a cellular receptor for Hepatitis A virus. An ortholog of TIM-1 was later identified in post-ischemic kidney tubules from rats and named kidney injury molecule-1 (KIM-1). It was found that patients with acute tubular nephritis secrete high levels of the protein into the urine. TIM-1 was subsequently cloned from cells in a mouse model of allergic asthma, suggesting a role in immune function. TIM-1 was later found to be expressed at elevated levels during the clinically inactive phase of multiple sclerosis, and accompanied by low expression of the inflammatory cytokine IFNγ indicating potential involvement in an anti-inflammatory function. As shown in the Examples herein, TIM-1 was found to be expressed on Bregs, and is required for Breg immunosuppressive function, Ligands for TIM-1 include TIM-4, which is expressed on antigen-presenting cells and some B cell subsets, and phosphatidyl serine.

The human polypeptide is expressed as a 359 amino acid precursor that includes a 20 amino acid signal sequence, a 270 amino acid extracellular domain, a 21 amino acid transmembrane region and a 48 amino acid cytoplasmic domain. The extracellular domain of TIM-1 comprises the ligand-binding domain of the receptor.

Sequences for TIM-1 are known for a number of species. The human TIM-1 NCBI Gene ID is 26762. Polynucleotide sequences (e.g. CCDS43392.1; coding sequence; SEQ ID NO: 1); mRNA sequence (e.g., NM_001173393.2; SEQ ID NO: 2) and polypeptide sequences (e.g. NP_01166864.1 SEQ ID DO: 3).

The following sequences of human TIM-1 are provided:

| SEQ ID NO: 1 >TIM-1 amino acid-encoding polynucleotide sequence, e.g. | |
|---|---|
| CCDS43392.1: | |
| ATGCATCCTCAAGTGGTCATCTTAAGCCTCATCCTACATCTGGCAGATTCTGTAGCTGGTTCTGTAAAGG | |
| TTGGTGGAGAGGCAGGTCCATCTGTCACACTACCCTGCCACTACAGTGGAGCTGTCACATCCATGTGCTG | |
| GAATAGAGGCTCATGTTCTCTATTCACATGCCAAAATGGCATTGTCTGGACCAATGGAACCCACGTCACC | |
| TATCGGAAGGACACACGCTATAAGCTATTGGGGGACCTTTCAAGAAGGGATGTCTCTTTGACCATAGAAA | |
| ATACAGCTGTGTCTGACAGTGGCGTATATTGTTGCCGTGTTGAGCACCGTGGGTGGTTCAATGACATGAA | |
| AATCACCGTATCATTGGAGATTGTGCCACCCAAGGTCACGACTACTCCAATTGTCACAACTGTTCCAACC | |
| GTCACGACTGTTCGAACGAGCACCACTGTTCCAACGACAACGACTGTTCCAATGACGACTGTTCCAACGA | |
| CAACTGTTCCAACAACAATGAGCATTCCAACGACAACGACTGTTCTGACGACAATGACTGTTTCAACGAC | |
| AACGAGCGTTCCAACGACAACGAGCATTCCAACAACAACAAGTGTTCCAGTGACAACAACTGTCTCTACC | |
| TTTGTTCCTCCAATGCCTTTGCCCAGGCAGAACCATGAACCAGTAGCCACTTCACCATCTTCACCTCAGC | |
| CAGCAGAAACCCACCCTACGACACTGCAGGGAGCAATAAGGAGAGAACCCACCAGCTCACCATTGTACTC | |
| TTACACAACAGATGGGAATGACACCGTGACAGAGTCTTCAGATGGCCTTTGGAATAACAATCAAACTCAA | |
| CTGTTCCTAGAACATAGTCTACTGACGGCCAATACCACTAAAGGAATCTATGCTGGAGTCTGTATTTCTG | |
| TCTTGGTGCTTCTTGCTCTTTTGGGTGTCATCATTGCCAAAAAGTATTTCTTCAAAAAGGAGGTTCAACA | |
| ACTAAGTGTTTCATTTAGCAGCCTTCAAATTAAAGCTTTGCAAAATGCAGTTGAAAAGGAAGTCCAAGCA | |
| GAAGACAATATCTACATTGAGAATAGTCTTTATGCCACGGACTAA | |
| SEQ ID NO: 2: TIM-1 mRNA sequence, e.g., NM_001173393.2 | |
| 1 | agtgctatta ctgcatatga tgtaggttta gttttccaag ttcttccgtg gccctttttg |
| 61 | cttattatat caatccttgg tgggagatag aggaagcatt tttagtgcta ttttacaact |
| 121 | gaggaaatag aggtttgaag agaactcagg aactctcagg gttacccagc attgtgagtg |
| 181 | acagagcctg gatctgaacg ctgatcccat aatgcatcct caagtggtca tcttaagcct |
| 241 | catcctacat ctggcagatt ctgtagctgg ttctgtaaag gttggtggag aggcaggtcc |
| 301 | atctgtcaca ctaccctgcc actacagtgg agctgtcaca tccatgtgct ggaatagagg |
| 361 | ctcatgttct ctattcacat gccaaaatgg cattgtctgg accaatggaa cccacgtcac |
| 421 | ctatcggaag gacacacgct ataagctatt gggggacctt tcaagaaggg atgtctcttt |
| 481 | gaccatagaa aatacagctg tgtctgacag tggcgtatat tgttgccgtg ttgagcaccg |
| 541 | tgggtggttc aatgacatga aaatcaccgt atcattggag attgtgccac ccaaggtcac |
| 601 | gactactcca attgtcacaa ctgttccaac cgtcacgact gttcgaacga gcaccactgt |
| 661 | tccaacgaca acgactgttc caatgacgac tgttccaacg acaactgttc caacaacaat |
| 721 | gagcattcca acgacaacga ctgttctgac gacaatgact gtttcaacga caacgagcgt |
| 781 | tccaacgaca acgagcattc caacaacaac aagtgttcca gtgacaacaa ctgtctctac |
| 841 | ctttgttcct ccaatgcctt tgcccaggca gaaccatgaa ccagtagcca cttcaccatc |
| 901 | ttcacctcag ccagcagaaa cccaccctac gacactgcag ggagcaataa ggagagaacc |
| 961 | caccagctca ccattgtact cttacacaac agatgggaat gacaccgtga cagagtcttc |
| 1021 | agatggcctt tggaataaca atcaaactca actgttccta gaacatagtc tactgacggc |
| 1081 | caataccact aaaggaatct atgctggagt ctgtatttct gtcttggtgc ttcttgctct |
| 1141 | tttgggtgtc atcattgcca aaaagtattt cttcaaaaag gaggttcaac aactaagtgt |
| 1201 | ttcatttagc agccttcaaa ttaaagcttt gcaaaatgca gttgaaaagg aagtccaagc |
| 1261 | agaagacaat atctacattg agaatagtct ttatgccacg gactaagacc cagtggtgct |
| 1321 | ctttgagagt ttacgcccat gagtgcagaa gactgaacag acatcagcac atcagacgtc |
| 1381 | ttttagaccc caagacaatt tttctgtttc agtttcatct ggcattccaa catgtcagtg |
| 1441 | atactgggta gagtaactct ctcactccaa actgtgtata gtcaacctca tcattaatgt |
| 1501 | agtcctaatt ttttatgcta aaactggctc aatccttctg atcattgcag ttttctctca |
| 1561 | aatatgaaca ctttataatt gtatgttctt tttagacccc ataaatcctg tatacatcaa |
| 1621 | agagaa |
| SEQ ID NO: 3 >TIM-1 polypeptide sequence as described by, e.g., NP_01166864.1: | |
| 1 | mhpqvvilsl ilhladsvag svkvggeagp svtlpchysg avtsmcwnrg scslftcqng |
| 61 | ivwtngthvt yrkdtrykll gdlsrrdvsl tientaysds gvyccrvehr gwfndmkitv |
| 121 | sleivppkvt ttpivttvpt vttvrtsttv pttttvpttt vpttmsiptt ttvpttmtvs |
| 181 | tttsvpttts iptttsvpvt ttvstfvppm plprqnhepv atspsspqpa ethpttlqga |
| 241 | irreptsspl ysyttdgndt vtessdglwn nnqtqlfleh slltanttkg iyagvcisvl |
| 301 | vllallgvii akkyffkkev qqlsvsfssl qikalqnave kevqaedniy ienslyatd |

TIGIT (T-Cell Immunoreceptor with Ig and ITIM Domains)

Human TIGIT is a 244 amino acid polypeptide having the amino acid sequence: (SEQ ID NO: 6), as described by, e.g., NP_776160.2. TIGIT polypeptides also include any naturally occurring allelic, splice variants, and processed forms thereof that function in promoting Breg immunosuppressive activity.

TIGIT is an immunoreceptor inhibitory checkpoint molecule. Expression of TIGIT has previously been demonstrated in both NK cells and T cells, and plays a role in their activation and maturation. Analogous to the activating receptor CD28/inhibitory receptor CTLA-4 pair, TIGIT competes with immunoactivator receptor CD226 (DNAM-1) for the same set of ligands: CD155 (PVR or poliovirus receptor) and CD112 (Nectin-2 or PVRL2).

Sequences for TIGIT are known for a number of species, e.g. human TIGIT (the TIGIT NCBI Gene ID is 201633), nucleotide sequences (e.g. CCDS2980.1 SEQ ID NO: 4), mRNA sequences (e.g., NM_173799.4; SEQ ID NO: 5) and polypeptide sequences (e.g. NP_776160.2 SEQ ID DO: 6).

The following sequences of human TIGIT are provided:

| SEQ ID NO: 4 >TIGIT nucleotide sequence as described by, e.g., CCDS2980.1: | |
|---|---|
| ATGCGCTGGTGTCTCCTCCTGATCTGGGCCCAGGGGCTGAGGCAGGCTCCCCTCGCCTCAGGAATGATGA | |
| CAGGCACAATAGAAACAACGGGGAACATTTCTGCAGAGAAAGGTGGCTCTATCATCTTACAATGTCACCT | |
| CTCCTCCACCACGGCACAAGTGACCCAGGTCAACTGGGAGCAGCAGGACCAGCTTCTGGCCATTTGTAAT | |
| GCTGACTTGGGGTGGCACATCTCCCCATCCTTCAAGGATCGAGTGGCCCCAGGTCCCGGCCTGGGCCTCA | |
| CCCTCCAGTCGCTGACCGTGAACGATACAGGGGAGTACTTCTGCATCTATCACACCTACCCTGATGGGAC | |
| GTACACTGGGAGAATCTTCCTGGAGGTCCTAGAAAGCTCAGTGGCTGAGCACGGTGCCAGGTTCCAGATT | |
| CCATTGCTTGGAGCCATGGCCGCGACGCTGGTGGTCATCTGCACAGCAGTCATCGTGGTGGTCGCGTTGA | |
| CTAGAAAGAAGAAAGCCCTCAGAATCCATTCTGTGGAAGGTGACCTCAGGAGAAAATCAGCTGGACAGGA | |
| GGAATGGAGCCCCAGTGCTCCCTCACCCCCAGGAAGCTGTGTCCAGGCAGAAGCTGCACCTGCTGGGCTC | |
| TGTGGAGAGCAGCGGGGAGAGGACTGTGCCGAGCTGCATGACTACTTCAATGTCCTGAGTTACAGAAGCC | |
| TGGGTAACTGCAGCTTCTTCACAGAGACTGGTTAG | |
| SEQ ID NO: 6 >TIGIT polypeptide sequence as described by, e.g., NP_776160.2: | |
| 1 | mrwcllliwa qglrqaplas gmmtgtiett gnisaekggs iilqchlsst taqvtqvnwe |
| 61 | qqdqllaicn adlgwhisps fkdrvapgpg lgltlqsltv ndtgeyfciy htypdgtytg |
| 121 | riflevless vaehgarfqi pllgamaatl vvictavivv valtrkkkal rihsvegdlr |
| 181 | rksagqeews psapsppgsc vqaeaapagl cgeqrgedca elhdyfnvls yrslgncsff |
| 241 | tetg |

Human TIGIT is expressed as a 244 amino acid precursor, with signal sequence amino acids 1-21, extracellular domain amino acids 22-141, transmembrane domain amino acids 142-162, and cytoplasmic domain amino acids 163-244. The 21 amino acid signal peptide sequence of TIGIT is underlined for reference. The sequence of human TIGIT, without the 21 amino acid signal peptide sequence, is provided below as:

(SEQ ID NO: 7)
MMTGTIETTGNISAEKGGSIILQCHLSSTTAQVTQVNWEQQDQLLAICN
ADLGWHISPSFKDRVAPGPGLGLTLQSLTVNDTGEYFCIYHTYPDGTYT
GRIFLEVLESSVAEHGARFQIPLLGAMAATLVVICTAVIVVVALTRKKK
ALRIHSVEGDLRRKSAGQEEWSPSAPSPPGSCVQAEAAPAGLCGEQRGE
DCAELHDYFNVLSYRSLGNCSFFTETG.

TIM-1 positively regulates expression of the immunosuppressive immunoreceptor TIGIT in Bregs, which is at least in part responsible for the immunosuppressive effects of TIM-1 expression.

Inhibitors of TIM-1 and TIGIT

Where TIM-1 and/or TIGIT promote the immunosuppressive effects of Bregs, methods and compositions described herein include inhibition of TIM-1 and/or TIGIT in Bregs or B cells to thereby promote and/or maintain anti-tumor or anti-infection immune activity.

Accordingly, provided herein are inhibitors of these regulators of B cell-mediated immunosuppression. Inhibitors of regulators of B cell-mediated immunosuppression can include, for example, an agent that binds specifically to the target regulator, such as TIM-1 and/or TIGIT, and inhibits its signaling activity. Thus, an inhibitor of TIM-1 and/or TIGIT activity in B cells can include a protein-binding agent that permits inhibition of TIM-1 and/or TIGIT signaling. Such agents include, but are not limited to, antibodies, protein-binding agents, multi-specific protein-binding agents, small molecules, recombinant proteins, peptides, aptamers, avimers and protein-binding derivatives, portions or fragments thereof. An agent that inhibits TIM-1 or TIGIT activity by binding to the target can, for example, bind to the extracellular domain of the target molecule—such binding can, for example, interfere with ligand binding and signal transduction by the receptor polypeptide. Agents that bind TIM-1 and/or TIGIT are described in further detail below.

Alternative agents that interfere with TIM-1 and/or TIGIT activity can also include, for example, soluble versions of the receptors themselves. Such constructs, generally comprising the extracellular domain of the receptor, either entirely without a transmembrane domain or lacking residues of the transmembrane domain sufficient to anchor it in the membrane, can bind receptor ligand(s) and thereby compete for ligand binding to the intact cell surface receptor. Soluble receptor constructs can be stabilized or maintained in circulation by, for example, expression as a fusion with a carrier such as an antibody Fc domain or serum albumin as well known to those of ordinary skill in the art.

Other inhibitors include agents that inhibit the expression of TIM-1 and/or TIGIT. Agents that inhibit the expression of TIM-1 and/or TIGIT can include, for example, RNA interference molecules or antisense RNA molecules that either direct the cleavage of the mRNA encoding the target factor or interfere with its translation, or both. The design of RNA interference (RNAi) molecules that target a given transcript is known to those of ordinary skill in the art, and RNAi molecules and nucleic acid constructs encoding them, e.g., constructs encoding shRNAs or miRNAs are also commercially available. This class of agents and methods for preparing and using them are all well-known in the art, as are ribozyme and miRNA molecules. See, e.g., PCT US2007/024067 (which is incorporated by reference herein in its entirety) for a thorough discussion.

In some embodiments, an inhibitor can be targeted to B cells. Methods for targeting an inhibitor to B cells include, for example, conjugating a molecule that specifically binds a cell surface marker exclusively or predominantly expressed on B cells, to the inhibitor. This approach can be used to target agents that inhibit expression of TIM-1 and/or TIGIT to a cell, e.g., by formulating an antibody or antigen-binding fragment thereof that binds a B cell-specific cell surface marker with an RNA interference agent or construct, e.g., in or on a liposome.

An alternative approach to targeting an agent to B cells includes conjugating the agent to a ligand that binds a B cell-specific cell surface marker. For example, Siglec-2 binds to the B cell-specific cell surface marker CD22 and can be conjugated to an agent that targets TIM-1 and/or TIGIT as described herein. CD21 is a co-receptor on the B cell receptor, and binds to antigen-bound C3d; thus conjugation of an agent that inhibits TIM-1 and/or TIGIT activity or expression to B cells to CD21 could provide B cell targeting of the agent.

An agent, such as a TIM-1- or TIGIT-binding agent can also be targeted to B cells by conjugation of the agent, including but not limited to an antibody reagent, to an antibody or antigen-binding fragment thereof that specifically binds a B cell-specific cell surface marker. Thus, in some embodiments, a B cell-targeted inhibitor comprises a bispecific antibody reagent. This bispecific design and others relevant to the claimed methods and compositions are discussed further herein below.

Antibody Inhibitors

As discussed above, in some embodiments, an inhibitory agent can comprise an antibody or antigen-binding fragment thereof that binds TIM-1 and/or TIGIT and inhibits their signaling activity. An “antibody reagent” is a polypeptide that includes at least one antigen-binding immunoglobulin variable domain sequence, and which specifically binds a given antigen (e.g., TIM-1 or TIGIT).

In one embodiment, an inhibitory antibody or antigen binding fragment thereof binds the TIM-1 or TIGIT extracellular domain and inhibits binding of natural ligands to the receptor molecule. Other mechanisms, such as interference with receptor interaction with other (co)regulatory molecules can also be effective; the key is that binding of the antibody reagent inhibits receptor signaling, and this can be verified in an appropriate cell culture assay.

A variety of suitable antibody reagent formats are known in the art, such as complete antibodies, e.g., an IgG, or modified forms or fragments of such antibodies, including, as non-limiting examples, single chain antibodies, heterodimers of antibody heavy chains and/or light chains, an Fv fragment (e.g., single chain Fv (scFv), a disulfide bonded Fv), a Fab fragment, a Fab′ fragment, a F(ab′)2 fragment), a single variable domain (e.g., V_{H}, VL, VHH), a dAb, and modified versions of any of the foregoing (e.g., modified by the covalent attachment of polyalkylene glycol (e.g., polyethylene glycol, polypropylene glycol, polybutylene glycol) or other suitable polymer). Antibody reagents or constructs can, if desired, be linked to an antibody Fc region, comprising one or both of CH2 and CH3 domains, and optionally, a hinge region. Such linkage can provide benefits such as increased serum half-life or promotion of effector function(s). Alternatively, antibody reagents or constructs can be fused to a carrier such as serum albumin to promote increased serum half-life.

In some embodiments, a polypeptide agent, including an antibody reagent, can be formatted as a bispecific polypeptide agent as described herein, and in US 2010/0081796 and US 2010/0021473, the contents of which are herein incorporated in their entireties by reference. Bispecific agents can include, for example, agents including separate binding sites specific for TIM-1 and a B cell-specific cell surface marker, TIGIT and a B cell-specific cell surface marker, or TIM-1 and TIGIT. In other embodiments, a polypeptide agent, including an antibody reagent, can be formatted as a multispecific polypeptide agent, for example as described in WO 03/002609, the entire teachings of which are incorporated herein by reference. The instance where, for example, an agent comprising binding sites for TIM-1, TIGIT and a B cell-specific cell surface marker is one example of a multispecific polypeptide agent.

Antibodies suitable for practicing the methods described herein are preferably monoclonal, and can include, but are not limited to, human, humanized or chimeric antibodies, including single chain antibodies, Fab fragments, F(ab′) fragments, fragments produced by a Fab expression library, and/or binding fragments of any of the above. Antibody reagents also include immunoglobulin molecules and immunologically active portions of immunoglobulin molecules, i.e., molecules that contain at least one, at least two, at least three or more antigen binding sites that specifically bind TIM-1 and/or TIGIT. Such immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule, as understood by one of skill in the art.

The term “monoclonal antibody” as used herein refers to an antibody produced by a single B cell clone, B cell hybrodima or its equivalent. Such a cell produces only one antibody, such that all antibodies produced by such a clone have the same antigen-binding domain. In contrast to polyclonal antibody preparations that typically include different antibodies directed against different determinants (epitopes) on a given target antigen, each monoclonal antibody is directed against a single determinant on the antigen. The term “monoclonal antibody” as used herein is not limited to antibodies produced through hybridoma technology. It is to be understood that the term “monoclonal antibody” refers to an antibody that is derived from a single clone, including any eukaryotic, prokaryotic or phage clone, and not the method by which the antibody is produced. For example, the monoclonal antibodies to be used in accordance with the methods and compositions described herein can be made by the hybridoma method first described by Kohler et al., Nature 256:495 (1975), or may be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example. A wide variety of methods for producing constructs with the antigen-binding domain of monoclonal antibodies are known to those of ordinary skill in the art.

Bispecific and multispecific polypeptide agents can comprise immunoglobulin variable domains that have different binding specificities. Such bispecific and multispecific polypeptide agents can comprise combinations of heavy and light chain domains. For example, a V_{H} domain and a VL domain, which can be linked together in the form of an scFv (e.g., using a suitable linker such as (Gly4Ser)n (where n=1-8, e.g., 2, 3, 4, 5, 6, or 7) (SEQ ID NO: 44) can provide one antigen-binding domain that binds one or each target of a multispecific polypeptide agent e.g. TIM-1, TIGIT and/or a B cell cell-surface target. A construct that includes, e.g., an scFv that binds TIM-1 and an scFv that binds TIGIT, is said to be bispecific for TIM-1 and/or TIGIT. Similar arrangements can be applied in the context of, e.g., a bispecific F(ab′)2 construct.

Single domain antibody constructs are also contemplated for the development of bispecific or multispecific reagents described herein. In some embodiments of the aspects described herein, the bispecific and multispecific polypeptide agents do not comprise complementary V_{H}/VL pairs which form an antigen-binding site that binds to a single antigen or epitope co-operatively as found in conventional two chain antibodies. Instead, in some embodiments, the bispecific and multispecific polypeptide agents can comprise a domain, wherein the V domains each have different binding specificities, such that two different epitopes or antigens are specifically bound. Camelid antibodies for example comprise only V_{H} domains, and can be used to generate bispecific constructs when modified to a humanized scaffold.

In addition, in some embodiments, bispecific and multispecific polypeptide agents comprise one or more CH or CL domains. A hinge region domain can also be included in some embodiments. Such combinations of domains can, for example, mimic natural antibodies, such as IgG or IgM, or fragments thereof, such as Fv, scFv, Fab or F(ab′) 2 molecules. Other structures, such as a single arm of an IgG molecule comprising V_{H}, VL, CH1 and CL domains, are also encompassed within the embodiments described herein. Alternatively, in another embodiment, a plurality of bispecific polypeptide agents is combined to form a multimer. An Fc domain that binds human FcRn can extend circulating half-life by directing internalized antibodies into the FcRn-mediated recycling/secretory pathway. As another alternative, fusion with serum albumin can also extend serum half-life.

It will be appreciated by one skilled in the art that the light and heavy variable regions of a bispecific or multispecific polypeptide agent produced according to the methods described herein can be on the same polypeptide chain, or alternatively, on different polypeptide chains. In the case where the variable regions are on different polypeptide chains, then they can be linked via a linker, generally a flexible linker (such as a polypeptide chain), a chemical linking group, or any other method known in the art.

A bispecific or multispecific polypeptide agent can be formatted using a suitable linker such as (Gly4Ser)n, where n=from 1 to 8, e.g., 2, 3, 4, 5, 6 or 7 (SEQ ID NO: 44). If desired, bispecific or multispecific polypeptide agents can be linked to an antibody Fc region, comprising one or both of CH2 and CH3 domains, and optionally a hinge region. For example, vectors encoding bispecific or multispecific polypeptide agents linked as a single nucleotide sequence to an Fc region can be used to prepare such polypeptides.

In another aspect, bispecific antibodies having an IgG-like format are provided. Such formats have the conventional four chain structure of an IgG molecule (2 heavy chains and two light chains), in which one antigen-binding region (comprised of a V_{H} and a VL domain) specifically binds TIM-1 and/or TIGIT and the other antigen-binding region (also comprised of a V_{H} and a VL domain) specifically binds a B cell-specific receptor. In some embodiments, each of the variable regions (2 V_{H} regions and 2 VL regions) is replaced with a dAb or single chain variable domain. The dAb(s) or single chain variable domain(s) that are included in an IgG-like format can have the same specificity or different specificities. In some embodiments, the IgG-like format is tetravalent and can have two, three or four specificities. For example, the IgG-like format can be bispecific and comprise 3 dAbs that have the same specificity and another dAb that has a different specificity; bispecific and comprise two dAbs that have the same specificity and two dAbs that have a common but different specificity; trispecific and comprise first and second dAbs that have the same specificity, a third dAb with a different specificity and a fourth dAb with a different specificity from the first, second and third dAbs; or tetraspecific and comprise four dAbs that each have a different specificity. Antigen-binding fragments of IgG-like formats (e.g., Fab, F(ab′)2, Fab′, Fv, scFv) can be prepared as is known to one of skill in the art, and as described herein.

In some embodiments of the aspects described herein, antigen-binding fragments of antibodies can be combined and/or formatted into non-antibody multispecific polypeptide structures to form multivalent complexes, which bind target molecules having the same epitope, thereby providing superior avidity. For example, natural bacterial receptors such as SpA can be used as scaffolds for the grafting of CDRs to generate ligands which bind specifically to one or more epitopes. Details of this procedure are described in U.S. Pat. No. 5,831,012, herein incorporated by reference in its entirety. Other suitable scaffolds include those based on fibronectin and affibodies. Details of suitable procedures are described in WO 98/58965, herein incorporated by reference in its entirety. Other suitable scaffolds are described in van den Beuken et al., J. Mol. Biol. 310:591-601 (2001), and scaffolds such as those described in WO 00/69907 (Medical Research Council), herein incorporated by reference in their entireties, which are based for example on the ring structure of bacterial GroEL or other chaperone polypeptides. In some embodiments, protein scaffolds can be combined.

In some embodiments of the aspects described herein, bispecific or multispecific polypeptide agents can be formatted as fusion proteins that contain a first antigen-binding domain that is fused directly to a second antigen-binding domain. If desired, in some embodiments, such a format can further comprise a half-life extending moiety. As noted above, an Fc domain that binds human FcRn can extend circulating half-life by directing internalized antibodies into the FcRn-mediated recycling secretory pathway. As another alternative, fusion with serum albumin can also extend serum half-life. The benefits of serum albumin binding can also be realized with an antigen-binding domain that binds serum albumin. For example, a multispecific polypeptide agent can comprise a first antigen-binding domain specific for TIM-1, that is fused to a second antigen-binding domain specific for TIGIT, and an antigen-binding domain that binds serum albumin.

Generally, the orientation of the polypeptide domains that have a binding site with binding specificity for a target, and whether a bispecific or multispecific polypeptide agent comprises a linker, are a matter of design choice. However, some orientations, with or without linkers, can provide better binding characteristics than other orientations. All orientations are encompassed by the aspects and embodiments described herein, and bispecific or multispecific polypeptide agents that contain an orientation that provides desired binding characteristics can be easily identified by screening.

In some embodiments of the aspects described herein, an inhibitor targets TIM-1 and/or TIGIT and a B cell-specific cell surface marker in a bi- or multispecific format, in order to inhibit or reduce expression or activity of TIM-1 and/or TIGIT in a B cell-targeted manner or specifically in B cells. As used herein, a “B cell-specific cell surface marker” refers to a molecule found on the surface of B cells that is not expressed or is expressed minimally in other cell populations, such as T cells. Non-limiting examples of B cell-specific surface receptors useful in the compositions and methods described herein include CD19, CD20, and CD22. Non-limiting examples of known therapeutic antibodies that can be used to provide at least one binding domain that binds to a B cell-specific cell-surface molecule include Rituximab (anti-CD20), Ofatumumab (anti-CD20), Ocrelizumab (anti-CD20), Veltuzumab (anti-CD20), MEDI-551 (anti-CD19), and Epratuzumab (anti-CD22).

In one embodiment, a bispecific or multispecific antibody reagent as described herein can utilize TIM-1 binding site sequences from monoclonal antibodies that specifically bind human TIM-1, including, but not limited to those obtained from, clone 3A12E10 (Abcam), clone 219211 (R&D systems), clone A-12 (Santa Cruz Biotechnology), and clone 3B3 (Bio X Cell). For example, an antigen binding site specific for TIM-1 having the CDRs of 3A12E10, and an antigen binding site specific for CD20 having the CDRs of the Veltuzumab antibody can be grafted onto an appropriate framework, such as a human IgG1 backbone, to generate a bispecific antibody construct which binds TIM-1 and is targeted to B cells.

In some embodiments of the aspects described herein, the binding sites of bispecific polypeptide agents, such as bispecific antibodies, are directed against a target's ligand interaction site. In other embodiments of the aspects described herein, the binding sites of the bispecific polypeptide agents are directed against a site on a target in the proximity of the ligand interaction site, in order to provide steric hindrance for the interaction of the target with its receptor or ligand. Preferably, the site against which antibody reagents or polypeptide agents as described herein are directed is such that binding of the target to its receptor or ligand is modulated, and in particular, inhibited or prevented.

Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule is usually done by affinity chromatography steps, but the product yields are low. Similar procedures are disclosed in WO 93/08829, and in Traunecker et al, EMBO J., 10:3655-3659 (1991), herein incorporated by reference in their entireties.

According to another approach, described, for example in WO96/27011, herein incorporated by reference in its entirety, the interface between a pair of antibody molecules can be engineered to maximize the percentage of heterodimers which are recovered from recombinant cell culture. Such interfaces can comprise at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

In one aspect, the bispecific antibodies described herein include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies can be made using any convenient cross-linking methods. Suitable cross-linking agents are well known in the art, and are disclosed in U.S. Pat. No. 4,676,980, along with a number of cross-linking techniques. In one embodiment, the bispecific antibodies do not comprise a heteroconjugate.

Techniques for generating bispecific antibodies from antibody fragments have also been described in the literature. For example, bispecific antibodies can be prepared using chemical linkage. For example, Brennan et al., Science, 229: 81 (1985) describe a procedure wherein intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. A bispecific antibody produced using this method can be used in any of the compositions and methods described herein.

Another option for production of bispecific or multispecific antibody reagents uses the dual variable domain immunoglobulin, or DvDIg approach. See, e.g., DiGiammarino et al., Meth. Mol. Biol. 899: 145-156 (2012). In this approach, each arm of the immunoglobulin molecule has two or more antigen-binding domains, which can be different, linked in tandem. The design has the benefit of providing bi- or multi-specificity without the problems generated by random assortment of differing light chains with differing heavy chains.

In some embodiments, bispecific antibodies for use in the compositions and methods described herein can be produced using any of the methods described in U.S. Patent Application No.: 20100233173; U.S. Patent Application No.: 20100105873; U.S. Patent Application No.: 20090155275; U.S. Patent Application No.: 20080071063; and U.S. Patent Application No.: 20060121042, the contents of each of which are herein incorporated in their entireties by reference. In some embodiments, a bispecific antibody specific for TIM-1 and/or TIGIT, TIM-1 and a B cell surface marker, or TIGIT and a B cell surface marker can be produced using any of the methods described in U.S. Patent Application No.: 20090175867 and U.S. Patent Application No.: 20110033483 the contents of which are herein incorporated in their entireties by reference.

In some embodiments, bispecific antibodies can be made by the direct recovery of Fab′-SH fragments recombinantly expressed, e.g., in E. coli, and chemically coupled to form bispecific antibodies. Chemical conjugation is based on the use of homo- and heterobifunctional reagents with E-amino groups or hinge region thiol groups. Homobifunctional reagents such as 5,5′-Dithiobis(2-nitrobenzoic acid) (DNTB) generate disulfide bonds between the two Fabs, and 0-phenylenedimaleimide (O-PDM) generate thioether bonds between the two Fabs (Brenner et al., 1985, Glennie et al., 1987). Heterobifunctional reagents such as N-succinimidyl-3-(2-pyridylditio) propionate (SPDP) combine exposed amino groups of antibodies and Fab fragments, regardless of class or isotype (Van Dijk et al., 1989). For example, Shalaby et al., J Exp. Med, 175: 217-225 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described, and can be used in the generation of bispecific antibodies. For example, bispecific antibodies have been produced using leucine zippers (Kostelny et al., J. Immunol, 148(5):1547-1553 (1992)). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (VL) by a linker which is too short to allow pairing between the two domains on the same chain. Accordingly, the V_{H} and VL domains of one fragment are forced to pair with the complementary V_{H} and VL domains of another fragment, thereby forming two antigen-binding sites. Another strategy for making bispecific antibody fragments by the use of single-chain Fv (sFv) dimers has also been reported. See Gruber et al., J. Immunol., 152:5368 (1994). Alternatively, the antibodies can be “linear antibodies” as described in Zapata et al. Protein Eng. 8(10):1057-1062 (1995). Briefly, these antibodies comprise a pair of tandem Fd segments (V_{H}-CH1-V_{H}-C H1) which form a pair of antigen binding regions. Linear antibodies can be bispecific or multispecific.

Antibodies useful in the present methods can be described or specified in terms of the particular CDRs they comprise. The compositions and methods described herein encompass the use of an antibody or derivative thereof comprising a heavy or light chain variable domain, where the variable domain comprises (a) a set of three CDRs, and (b) a set of four framework regions, and in which the antibody or antibody derivative thereof specifically binds TIM-1, TIGIT or a B cell-specific cell surface marker.

Also provided herein are chimeric antibody derivatives of the bispecific and multispecific polypeptide agents, i.e., antibody molecules in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Chimeric antibody molecules can include, for example, one or more antigen binding domains from an antibody of a mouse, rat, or other species, with human constant regions. A variety of approaches for making chimeric antibodies have been described and can be used to make chimeric antibodies containing the immunoglobulin variable region which recognizes the selected antigens, on the surface of differentiated cells or tumor-specific cells. See, for example, Takeda et al., 1985, Nature 314:452; Cabilly et al., U.S. Pat. No. 4,816,567; Boss et al.; Tanaguchi et al., European Patent Publication EP171496; European Patent Publication 0173494, United Kingdom patent GB 2177096B).

The bispecific and multispecific polypeptide agents described herein can also include humanized antibody derivatives. Humanized forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments, antibodies described herein include derivatives that are modified, i.e., by the covalent attachment of another type of molecule to the antibody that does not prevent the antibody from binding to its target. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids.

Bispecific or multispecific antibodies as described herein can be generated by any suitable method known in the art. Monoclonal and polyclonal antibodies against TIM-1, TIGIT and B cell-specific cell-surface markers are known in the art. To the extent necessary, e.g., to generate antibodies with particular characteristics or epitope specificity, the skilled artisan can generate new monoclonal or polyclonal anti-TIM-1, anti-TIGIT and anti-B cell-specific cell-surface markers antibodies as discussed below or as known in the art. In other embodiments, the bispecific and multispecific antibodies and antigen-binding fragments thereof described herein can utilize TIM-1 binding site sequences from monoclonal antibodies against human TIM-1, such as, human monoclonal anti-TIM-1 IgG2 antibodies 1.29; 2.56.2; 2.59.2; and 2.45.1 (US2005/0084449); or those obtained from, clone 3A12E10 (Abcam), clone 219211 (R&D systems), clone A-12 (Satnta Cruz Biotechnology), clone 3B3 (Bio X Cell). Similarly, the bispecific and multispecific antibodies and antigen-binding fragments thereof described herein can utilize TIGIT binding site sequences from monoclonal antibodies against human TIGIT, such as those obtained from clone BMS-986207 (Bristol Myers Squibb), or clone MBSA43 (Thermo Fisher Scientific). For example, an antigen binding site against TIM-1 having the amino acid sequences of the CDR regions of 3A12E10, and/or an antigen binding site against TIGIT having the amino acid sequences of the CDR regions of the antibody produced by clone BMS-986207 can be grafted onto an appropriate framework, such as a human IgG1 backbone with the antigen-binding sequence for a B-cell specific cell-surface molecule, to generate a bispecific antibody construct as described herein.

Monoclonal antibodies can be prepared using a wide variety of techniques known in the art including the use of hybridoma, recombinant, and phage display technologies, or a combination thereof. Various methods for making monoclonal antibodies described herein are available in the art. For example, monoclonal antibodies can be produced using hybridoma techniques including those known in the art and taught, for example, in Harlow et al., Antibodies: A Laboratory Manual, (Cold Spring Harbor Laboratory Press, 2nd ed., 1988); Hammerling, et al., in: Monoclonal Antibodies and T-Cell Hybrido-mas 563-681 (Elsevier, N.Y., 1981) (said references incorporated by reference in their entireties). A wide range of other approaches are known to those of skill in the art.

RNA Interference (RNAi)

Other approaches for inhibiting TIM-1 and/or TIGIT expression and/or activity include the use of RNA interference (RNAi) or other approaches to specifically inhibit expression of TIM-1 and/or TIGIT proteins or aptamers as well as the use of small molecules or aptamers to inhibit the activity of TIM-1 and/or TIGIT. For each of these, it is contemplated that targeting to B cells can be achieved, for example, by conjugating the inhibitor to an aptamer that binds a B cell specific cell surface molecule. Alternatively, liposomes comprising the RNAi, aptamers or small-molecule can be designed to display B cell-specific cell-surface binding; molecules, e.g. aptamer or antibody binding domains on their surface to target delivery to B cells. The design and testing of RNAi molecules that inhibit the expression of TIM-1 and/or TIGIT are known to those skilled in the art. For example, RNAi molecules that inhibit TIM-1 include those from Santa Cruz Biotechnology, Inc. (Catalog no. sc-61691) and are also described by Kondratowicz et al., Proc Natl Acad Sci USA. 2011. RNAi molecules that inhibit TIGIT include those obtained from Dharmacon (Catalog no. A-018488-16-0005) and Thermo Fisher (Catalog no. AM16708).

The iRNA can be siRNA, shRNA, endogenous microRNA (miRNA), or artificial miRNA. In one embodiment, an iRNA as described herein effects inhibition of the expression and/or activity of a target, e.g. TIM-1 and/or TIGIT. In some embodiments of any of the aspects, the agent is siRNA that inhibits TIM-1 and/or TIGIT activity and/or expression.

One skilled in the art can design siRNA, shRNA, or miRNA to target TIM-1 and/or TIGIT, e.g., using publically available design tools. siRNA, shRNA, or miRNA can be synthetically made or expressed from a vector. Commercial sources include companies such as Dharmacon (Lafayette, Colo.) and Sigma Aldrich (St. Louis, Mo.), among others. Non-limiting examples of siRNA and shRNA molecule inhibitors of TIM-1 include TIM-1 siRNA and shRNA plasmids (Santa Cruz Biotechnology), among others. Non-limiting examples of siRNA and shRNA molecule inhibitors of TIGIT include TIGIT siRNA and shRNA plasmids (Santa Cruz Biotechnology), among others.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the methods and compositions described herein can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

In one embodiment, the agent is miRNA that inhibits TIM-1 and/or TIGIT expression and/or activity. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be encoded by a nucleic acid that is expressed in the cell, e.g., from naked DNA, or can be encoded by a nucleic acid that is contained within a vector.

The agent may result in gene silencing of the target gene (e.g., TIM-1 and/or TIGIT), such as with an RNAi molecule (e.g. siRNA or miRNA). This entails a decrease in the mRNA level in a cell for a target by at least about 5%, at least about 10%, at least about 20%, at least about 30%, at least about 40%, at least about 50%, at least about 60%, at least about 70%, at least about 80%, at least about 90%, at least about 95%, at least about 99%, or even about 100% (i.e., below detectable limits by standard miRNA assay detection methods) of the mRNA level found in the cell without the presence of the agent. One skilled in the art will be able to readily assess whether the siRNA, shRNA, or miRNA effectively targets e.g., TIM-1 and/or TIGIT, for downregulation, for example by transfecting the siRNA, shRNA, or miRNA into cultured cells and detecting the levels of a gene product (e.g., TIM-1 and/or TIGIT) found within the cell via western-blotting.

The agent can be contained in or expressed by a desired vector. Many such vectors useful for transferring exogenous genes into target mammalian cells are available. As used herein, a vector can be viral or non-viral. The term “vector” encompasses any genetic element that is capable of replication when associated with the proper control elements and that can transfer gene sequences to cells. A vector can include, but is not limited to, an expression vector, a plasmid, phage, transposon, cosmid, artificial chromosome, virus, virion, etc.

An expression vector can direct expression of an RNA or polypeptide (e.g., a TIM-1 and/or TIGIT inhibitor) from nucleic acid sequences contained therein linked to transcriptional regulatory sequences on the vector. The sequences expressed will often, but not necessarily, be heterologous to the cell. An expression vector can comprise additional elements, for example, the expression vector may have two replication systems, thus allowing it to be maintained in two organisms, for example in human cells for expression and in a prokaryotic host for cloning and amplification. Expression refers to the cellular processes involved in producing RNA and/or proteins and as appropriate, secreting proteins, including where applicable, but not limited to, for example, transcription, transcript processing, translation and protein folding, modification and processing. “Expression products” include RNA transcribed from a gene and processing derivatives thereof, such as siRNA, shRNA, miRNA, etc., and polypeptides obtained by translation of mRNA transcribed from a gene or gene construct.

Vectors can be episomal, e.g. plasmids, virus-derived vectors such as cytomegalovirus, adenovirus, etc., or can be integrated into the target cell genome, through homologous recombination or random integration, e.g. for retrovirus-derived vectors such as MMLV, HIV-1, ALV, etc. In some embodiments, combinations of retroviruses and an appropriate packaging cell line may also find use, where the capsid proteins will be functional for infecting the target cells. Commonly used retroviral vectors are “defective”, i.e. unable to produce viral proteins required for productive infection. Replication of the vector requires growth in the packaging cell line.

Integrating vectors, such as retroviral vectors, lentiviral vectors, hybrid adenoviral vectors, and herpes simplex viral vector are specifically contemplated for use in the methods described herein. Alternatively, non-integrative vectors (e.g., non-integrative viral vectors) can be used and can eliminate the risks posed by integrative retroviruses, as they do not incorporate their genome into the host DNA. Non-limiting examples of non-integrating viral vectors include Epstein Barr oriP/Nuclear Antigen-1 (“EBNA1”) vector, RNA Sendai viral vector, or an F-deficient Sendai virus vector. Another example of a non-integrative vector is a minicircle vector. Minicircle vectors are circularized vectors in which the plasmid backbone has been released leaving only the eukaryotic promoter and cDNA(s) that are to be expressed.

Viral vectors can also be targeted, e.g. to B cells by manipulating the viral capsid to comprise or display a ligand for a B cell-specific cell-surface molecule as known in the art.

Liposomes

An RNAi molecule preparation targeting TIM-1 and/or TIGIT can be formulated for delivery in a membranous molecular assembly, e.g., a liposome or a micelle. Targeted liposomal drug delivery systems for the treatment of B cell malignancies have been documented (see, e.g. Mittal et al., J Drug Target. 2014 June; 22(5):372-86.). A liposome is a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the siRNA composition. The lipophilic material isolates the aqueous interior from an aqueous exterior, which typically does not include the siRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the dsRNA are delivered into the cell where the dsRNA can specifically bind to a target RNA and can mediate RNAi. In some cases the liposomes are also specifically targeted, e.g., to direct a dsRNA, other RNAi molecule or small molecule to particular cell types.

A liposome containing e.g. a siRNA molecule can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The dsRNA preparation is then added to the micelles that include the lipid component. The cationic groups on the lipid interact with the siRNA and condense around the dsRNA to form a liposome. After condensation, the detergent is removed, e.g., by dialysis, to yield a liposomal preparation of the RNAi molecule.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety). These methods are readily adapted to packaging siRNA preparations into liposomes.

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

In some embodiments, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Felgner, P. L. et al., Proc. Natl. Acad. Sci., USA 8:7413-7417, 1987 and U.S. Pat. No. 4,897,355 for a description of DOTMA and its use with DNA, which are incorporated by reference in their entirety).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Small Molecule Inhibitors

In some embodiments, the inhibitors of TIM-1 and/or TIGIT are small molecules. As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Agents can be known to have a desired activity and/or property, or can be identified from a library of diverse compounds. Methods for screening small molecules are known in the art and can be used to identify a small molecule that is effective at, for example, inhibition of TIM-1 and/or TIGIT activity and/or expression.

Non-limiting examples of small molecule inhibitors of TIGIT include MK-7684 (and derivatives thereof) (Merck Sharp & Dohme Corp.).

Checkpoint Inhibitors

Among the most promising approaches to activating therapeutic anti-tumor immunity is the blockade of immune checkpoints. Immune-checkpoints refer to a network of inhibitory pathways hardwired into the immune system that are crucial for maintaining self-tolerance and modulating the duration and amplitude of physiological immune responses in peripheral tissues, in order to minimize collateral tissue damage. The immune systems of tumor patients or at least a tumor's microenvironment tend to have excessive inhibitory functions, which are induced or mediated by regulatory T cells (Tregs), regulatory B cells (Bregs), myeloid-derived suppressor cells (MDSCs), or the secretion of immunosuppressive cytokines, such as transforming growth factor-β (TGF-β) and interleukin-10 (IL-10). These conditions constitute an extremely favorable microenvironment for tumor progression. Checkpoint inhibitor therapies, which ‘unblock’ an existing immune response, or which unblock the initiation of an immune response, are very effective at treating cancer in a subgroup of subjects and tumor types. Since many of the immune checkpoints are regulated by interactions between specific receptor and ligand pairs, monoclonal antibodies, soluble receptor forms or other agents can be used to block such interactions and prevent or reverse immunosuppression. TIGIT, which is discussed herein above, is a checkpoint receptor molecule targeted for inhibition in B cells by methods and compositions as described herein. The two checkpoint receptors that have received the most attention in recent years are Cytotoxic T-Lymphocyte Associated protein 4 (CTLA-4) and PD-1 (Programmed Cell Death 1), but others are also important, including but not limited to TIM-3 and LAG-3. Each of these checkpoint molecules is contemplated as a therapeutic target in combination with an agent that targets TIM-1 and/or TIGIT in B cells for the treatment of cancer or chronic infection as described herein. In one embodiment an inhibitor of one or more of these additional checkpoints is administered in combination with an agent that targets TIM-1 and/or TIGIT in B cells. In another embodiment, the additional checkpoint inhibitor is also targeted to B cells, e.g., by conjugation or fusion with an antibody or antigen-binding domain thereof that binds a B cell-specific cell surface marker as described herein. The following provides additional detail regarding these additional checkpoint receptors and inhibitors thereof.

T-cell immunoglobulin and mucin-domain containing-3 or TIM-3 was initially identified molecule selectively expressed on IFNg-producing CD4+ Th1 and CD8+ Tc1 cells, and blockade of TIM-3 was shown to exacerbate experimental autoimmune encephalomyelitis (EAE), an animal model of multiple sclerosis, thereby implicating TIM-3 in the regulation of tolerance. A natural ligand for TIM-3 was identified as galectin 9. TIM-3 has subsequently been shown to be an immune checkpoint receptor, expressed for example, on dysfunctional or exhausted tumor-associated T cells and expressed by a variety of different cancers. Inhibition of TIM-3 has slowed tumor progression in animal models including models of colon carcinoma, colon adenocarcimoma, prostate cancer and WT3 sarcoma, among others.

TIM-3 sequences are known for a number of species. For example, the NCBI GeneID for human TIM-3 is 84868. The polynucleotide sequence for human TIM-3 mRNA is available at, for example, GenBank Accession No. JX049979.1, and the polypeptide sequence for human TIM-3 is available at GenBank Accession No. AFO66593.1, each of which is incorporated herein by reference.

Inhibitors of TIM-3 include antibodies and constructs including antigen-binding domains thereof that specifically bind TIM-3, e.g., antibodies and antigen-binding domains thereof that specifically bind the TIM-3 extracellular domain. Examples include the humanized monoclonal MGB453 (Novartis) and monoclonal TSR-022 (Tesaro), which are both in human clinical trials. TIM-3 antibodies are described, for example, in WO2015117002 and U.S. Pat. No. 9,605,070, the description of both of which in regard to anti-TIM-3 antibodies are incorporated herein by reference.

Cytotoxic T-lymphocyte Associated protein 4 (CTLA-4) is an immune checkpoint protein that downregulates pathways of T-cell activation (Fong et al., Cancer Res. 69(2):609-615, 2009; Weber Cancer Immunol. Immunother, 58:823-830, 2009). Human CTLA-4 (Cytotoxic T-Lymphocyte Associated protein 4) is a 223 amino acid polypeptide including its signal sequence. CTLA-4 polypeptides include that full length polypeptide, homologues in different species, as well as the processed forms lacking the signal sequence and any naturally occurring allelic, splice variants, and processed forms thereof that retain activity as an inhibitory immune checkpoint receptor. Typically, CTLA-4 refers to human CTLA-4.

Polynucleotide and polypeptide sequences for CTLA-4 are known for a number of species. The NCBI Gene ID for human CTLA-4 is 1493. Polynucleotide sequences include the genomic DNA sequence (e.g. NC_000002.12), and mRNA sequence (e.g., NM_001037631.3), which encodes the polypeptide sequence (e.g. GenBank Accession No. P16410.3), each of which is incorporated herein by reference.

Blockade of CTLA-4 has been shown to augment T-cell activation and proliferation. Inhibitors of CTLA-4 include anti-CTLA-4 antibodies, which, for example, bind to CTLA-4 and block its interaction with its ligands CD80/CD86 expressed on antigen presenting cells. Such inhibitors, block the down regulation of the immune responses mediated by the interaction of CTLA-4 with its ligands. Examples of anti-CTLA-4 antibodies are described in U.S. Pat. Nos. 5,811,097; 5,811,097; 5,855,887; 6,051,227; 6,207,157; 6,682,736; 6,984,720; and 7,605,238, the teachings of which regarding antibodies and antibody sequences are incorporated herein by reference. One anti-CTLA-4 antibody is tremelimumab, (ticilimumab, CP-675,206). In one embodiment, the anti-CTLA-4 antibody is ipilimumab (also known as 10D1, MDX-D010), a fully human monoclonal IgG antibody that binds to CTLA-4. Ipilimumab is marketed under the name YERVOY™ and has been approved for the treatment of unresectable or metastatic melanoma.

PD-1 is an immune checkpoint protein that limits the activity of T cells in peripheral tissues at the time of an inflammatory response to infection and limits autoimmunity. PD-1 blockade in vitro enhances T-cell proliferation and cytokine production in response to a challenge by specific antigen targets or by allogeneic cells in mixed lymphocyte reactions. A strong correlation between PD-1 expression and T cell response was shown with blockade of PD-1 (Pardoll, Nature Reviews Cancer, 12: 252-264, 2012).

Human PD-1 is a 288 amino acid polypeptide including its signal sequence. PD-1 polypeptides include that full length polypeptide, homologues in different species, as well as the processed forms lacking the signal sequence and any naturally occurring allelic, splice variants, and processed forms thereof that retain activity as an inhibitory immune checkpoint receptor. Sequences for PD-1 are known for a number of species. For example, the human PD-1 NCBI Gene ID is 5133. Human PD-1 mRNA sequence is found at GenBank Accession No. NM_005018.3, and the human polypeptide sequence is found at GenBank Accession No. NP_005009.2; each of these sequences is incorporated herein by reference.

PD1 blockade can be accomplished by a variety of mechanisms including, for example, administration of antibodies that bind PD-11 or its ligand, PD-L1 and inhibit suppressive signaling. Examples of PD-1 and PD-L1 blockers are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699, the description of each of which regarding anti-PD-1 antibodies and antibody sequences is incorporated herein by reference. Examples of anti-PD-1 antibodies and similar inhibitors include: nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody that binds PD-1; CT-011 a humanized antibody that binds PD-1; AMP-224, a fusion protein of the extracellular domain of PD-1 ligand PD-L2 and the Fc region of human IgG1; BMS-936559 (MDX-1105-01), and antibody specific for PD-L1 (B7-H1) blockade.

Lymphocyte Activation Gene 3 (LAG-3; CD223) is an immune checkpoint molecule expressed on the surface of activated T cells, NK cells, B cells and plasmacytoid dendritic cells. Human LAG-3 is a 525 amino acid polypeptide including its signal sequence. LAG-3 polypeptides include that sequence, as well as homologues in other species together with any naturally occurring allelic, splice variants, and processed forms thereof that retain activity as an inhibitory immune checkpoint receptor. Polynucleotide and polypeptide sequences for LAG-3 are known for a number of species; the human LAG-3 NCBI Gene ID is 3902; human LAG-3 mRNA sequence is available at GenBank Accession No. X51985.3; and human LAG-3 polypeptide sequence is available at GenBank Accession No. NP_002277.4, each of which are incorporated herein by reference.

Blockade of LAG-3 can be achieved using, for example, antibodies or constructs including the antigen-binding domains thereof, as well as soluble forms of the receptor. Antibodies to LAG-3 are known, including, for example: BMS-986016 (Creative Biolabs Cat. CBMAB-062LC), a human IgG that specifically binds human LAG-3; LAG3.5 (Creative Biolabs Cat. HPAB-0061-WJ), a human IgG4 that specifically binds human LAG-3; and a number of human monoclonal anti-LAG-3 antibodies described in WO2010019570, which is incorporated herein by reference. Other LAG-3 inhibitors include, for example, IMP321, a soluble Ig fusion with the extracellular domain of LAG-3 (Brignone et al., 2007, J. Immunol. 179:4202-4211).

Ectonucleoside triphosphate diphosphohydrolase-1 (ENTPD1; CD39) is an immune checkpoint molecule that catalyses the hydrolysis of γ- and β-phosphate residues of triphospho- and diphosphonucleosides to the monophosphonucleoside derivative. CD39 hydrolyzes adenosine triphosphate (ATP) and adenosine diphosphate (ADP) into adenosine monophosphate (AMP), which is then processed into adenosine by the CD73 ecto-5′-nucleotidas (Nt5e).

Human CD39 is a 522 amino acid polypeptide. CD39 polypeptides include that sequence, as well as homologues in other species together with any naturally occurring allelic, splice variants, and processed forms thereof that retain activity as an inhibitory immune checkpoint molecule. Polynucleotide and polypeptide sequences for CD39 are known for a number of species; the human CD39 NCBI Gene ID is 953; human CD39 mRNA sequence is available at GenBank Accession No. NM_001164178.1; and human CD39 polypeptide sequence is available at GenBank Accession No. NP_001157650.1, each of which are incorporated herein by reference.

Leukocyte antigen (CD73) is an immune checkpoint molecule that is encoded by the CD73 gene. The protein encoded by this gene is a member of the transmembrane 4 superfamily, also known as the tetraspanin family. Most of these members are cell-surface proteins that are characterized by the presence of four hydrophobic domains. The proteins mediate signal transduction events that play a role in the regulation of cell development, activation, growth and motility. This encoded protein is a cell surface glycoprotein that is known to complex with integrins and other transmembrane 4 superfamily proteins.

Human CD73 is a 213 amino acid polypeptide. CD73 polypeptides include that sequence, as well as homologues in other species together with any naturally occurring allelic, splice variants, and processed forms thereof that retain activity as an inhibitory immune checkpoint molecule. Polynucleotide and polypeptide sequences for CD39 are known for a number of species; the human CD73 NCBI Gene ID is 951; human CD73 mRNA sequence is available at GenBank Accession No. NM_001040031.1; and human CD73 polypeptide sequence is available at GenBank Accession No. NP_001035120.1, each of which are incorporated herein by reference.

Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). It is contemplated that B7 inhibitors can also provide a benefit in combination with TIM-1 and/or TIGIT inhibitors in B cells as described herein for the treatment of diseases or disorders involving inappropriate suppression of B cell immune activity, such as cancer and chronic infection.

Methods of Treatment

In some aspects, provided herein are methods of treating a disease or disorder involving inappropriate immunosuppression, the method comprising administering a therapeutically effective amount of an agent that decreases the expression or activity of TIM-1 in B cells to a subject in need thereof. In other aspects, provided herein are methods of treating a disease or disorder involving inappropriate immunosuppression, the method comprising administering a therapeutically effective amount of an agent that decreases the expression or activity of TIGIT in B cells to a subject in need thereof. In other aspects, provided herein are methods of treating a disease or disorder involving inappropriate immunosuppression, the method comprising administering a therapeutically effective amount of an agent that decreases the expression or activity of TIM-1 and the expression or activity of TIGIT in B cells to a subject in need thereof. In these aspects, the agent that decreases expression or activity of TIM-1 and/or TIGIT can be targeted to B cells.

It is specifically contemplated that the inhibitor of TIM-1 and/or TIGIT as described herein can be administered with one or more additional anti-cancer therapies. In such instances, the inhibitor of TIM-1 and/or TIGIT can be administered simultaneously with the additional anti-cancer therapy, in the same or in separate compositions, or sequentially. For sequential administration, the TIM-1 and/or TIGIT inhibitory agent as described herein can be administered first, and the additional agent can be administered second, or the order of administration can be reversed. The agent and/or other therapeutic agents, procedures or modalities can be administered during periods of active disease, or during a period of remission or less active disease. The agent(s) as described herein can be administered before another treatment, concurrently with the treatment, post-treatment, or during remission of the disorder.

Also provided herein are methods of reducing B-cell-mediated immunosuppression in a subject comprising administering an inhibitor of TIM-1 and/or TIGIT activity or expression in B cells to a subject in need thereof.

Accordingly, in some embodiments of these methods and all such methods described herein, the disease or disorder involving inappropriate immunosuppression to be treated or prevented using the methods described herein, include, but are not limited to cancer and chronic infection.

Cancer

In some embodiments of these aspects and all such aspects described herein, the subject in need thereof has or has been diagnosed with cancer.

In certain embodiments, the cancer is metastatic. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Metastases are most often detected through the sole or combined use of magnetic resonance imaging (MM) scans, computed tomography (CT) scans, blood and platelet counts, liver function studies, chest X-rays and bone scans in addition to the monitoring of specific symptoms.

Examples of cancer include but are not limited to, carcinoma, lymphoma, blastoma, sarcoma, and leukemia. More particular examples of such cancers include, but are not limited to basal cell carcinoma, biliary tract cancer; bladder cancer; bone cancer; brain and CNS cancer; breast cancer; cancer of the peritoneum; cervical cancer; cholangiocarcinoma; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; teratocarcinoma; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), tumors of primitive origins and Meigs' syndrome.

In some embodiments of these methods and all such methods described herein, the methods further comprise administering an anti-cancer therapy or agent to a subject in addition to the inhibitor of TIM-1 and/or TIGIT activity or expression in B cells. In some embodiments, inhibitors of TIM-1 and/or TIGIT are administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

The term “anti-cancer therapy” refers to a therapy useful in treating cancer other than the TIM-1 and/or TIGIT targeting therapeutic disclosure disclosed herein. Examples of anti-cancer therapeutic agents include, but are not limited to, e.g., surgery, chemotherapeutic agents, growth inhibitory agents, cytotoxic agents, agents used in radiation therapy, anti-angiogenesis agents, apoptotic agents, anti-tubulin agents, and other agents to treat cancer, such as anti-HER2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX2 inhibitor (e.g., celecoxib), interferons, cytokines, antagonists (e.g., neutralizing antibodies) that bind to one or more of the following targets: other TIM family members (e.g. TIM-3), CEACAM1 or any CEACAM family member, ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also specifically contemplated for the methods described herein.

In some embodiments, an anti-cancer therapy comprises an immunotherapy such as adoptive cell transfer. “Adoptive cell transfer,” as used herein, includes immunotherapies involving genetically engineering a subject or patient's own T cells to produce special receptors on their surface called chimeric antigen receptors (CARs). CARs are proteins that allow the T cells to recognize a specific protein (antigen) on tumor cells. These engineered CAR T cells are then grown in the laboratory until they number in the billions. The expanded population of CAR T cells is then infused into the patient. After the infusion, the T cells multiply in the subject's body and, with guidance from their engineered receptor, recognize and kill cancer cells that harbor the antigen on their surfaces.

Cytotoxic agents include, for example, radioactive isotopes (e.g. At²¹¹, I¹¹³, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including active fragments and/or variants thereof.

In some embodiments of these methods and all such methods described herein, the methods further comprise administering a chemotherapeutic agent to the subject being administered the inhibitor of TIM-1 and/or TIGIT activity or expression in B cells.

Non-limiting examples of chemotherapeutic agents can include include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB.); inhibitors of PKC-alpha, Raf, H-Ras, EGFR (e.g., erlotinib (TARCEVA®)) and VEGF-A that reduce cell proliferation and pharmaceutically acceptable salts, acids or derivatives of any of the above. In addition, the methods of treatment can further include the use of radiation or radiation therapy.

Chemotherapeutic agents as used herein encompass both chemical and biological agents. These agents function, for example, to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; Baltzer L, Berkery R (eds): Oncology Pocket Guide to Chemotherapy, 2nd ed. St. Louis, Mosby-Year Book, 1995; Fischer D S (ed): The Cancer Chemotherapy Handbook, 4th ed. St. Louis, Mosby-Year Book, 2003)).

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

In some embodiments of these methods and all such methods described herein, the methods further comprise administering a tumor or cancer antigen to a subject being administered inhibitor of TIM-1 and/or TIGIT activity or expression in B cells.

A number of tumor antigens have been identified that are associated with specific cancers. As used herein, the terms “tumor antigen” and “cancer antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER2, mucins (i.e., MUC-1), prostate-specific antigen (PSA), and prostatic acid phosphatase (PAP). In addition, viral proteins such as hepatitis B (HBV), Epstein-Barr (EBV), and human papilloma (HPV) have been shown to be important in the development of hepatocellular carcinoma, lymphoma, and cervical cancer, respectively. However, due to the immunosuppression of patients diagnosed with cancer, the immune systems of these patients often fail to respond to the tumor antigens.

By “reduce” or “inhibit” in terms of the cancer treatment methods described herein is meant the ability to cause an overall decrease preferably of 20% or greater, 30% or greater, 40% or greater, 45% or greater, more preferably of 50% or greater, of 55% or greater, of 60% or greater, of 65% or greater, of 70% or greater, and most preferably of 75% or greater, 80% or greater, 85% or greater, 90% or greater, or 95% or greater, for a given parameter or symptom. Reduce or inhibit can refer to, for example, the presence or size of metastases or micrometastases, the size of the primary tumor, the presence or the size of the dormant tumor, etc. A patient or subject who is being treated for a cancer or tumor is one who a medical practitioner has diagnosed as having such a condition. Diagnosis can be by any suitable means.

Chronic Infection

In some embodiments of these aspects and all such aspects described herein, the subject in need thereof has or has been diagnosed with a chronic infection.

In a “chronic infection,” the infectious agent is present in the subject at all times. However, the signs and symptoms of the disease can be present or absent for an extended period of time. Non-limiting examples of chronic infection include hepatitis B (caused by hepatitis B virus (HBV)) and hepatitis C (caused by hepatitis C virus (HCV)) adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyomavirus BK, polyomavirus JC, measles virus, rubella virus, human immunodeficiency virus (HIV), human T cell leukemia virus I, and human T cell leukemia virus II. Parasitic persistent infections can arise as a result of infection by, for example, Leishmania, Toxoplasma, Trypanosoma, Plasmodium, Schistosoma, and Encephalitozoon.

In some embodiments, a chronic infection can be a latent infection. In some embodiments, a chronic infection can include periods in which the infection is a latent infection. In a “latent infection,” the infectious agent (such as a virus) is seemingly inactive and dormant such that the subject does not always exhibit signs or symptoms. In a latent viral infection, the virus remains in equilibrium with the host for long periods of time before symptoms again appear; however, the actual viruses cannot typically be detected until reactivation of the disease occurs. Non-limiting examples of latent infections include infections caused by herpes simplex virus (HSV)-1 (fever blisters), HSV-2 (genital herpes), and varicella zoster virus VZV (chickenpox-shingles).

Dosage, Administration, Efficacy

Dosage

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions, methods, and uses that exhibit large therapeutic indices are preferred. A therapeutically effective dose can be estimated initially from cell culture assays. Also, a dose can be formulated in animal models to achieve a circulating plasma concentration range that includes the IC50, which achieves a half-maximal inhibition of measured function or activity as determined in cell culture, or in an appropriate animal model. The effects of any particular dosage can be monitored by a suitable bioassay. The dosage can be determined by a physician and adjusted, as necessary, to suit observed effects of the treatment.

Administration

The agents described herein can be administered to a subject in need thereof by any appropriate route which results in an effective treatment in the subject.

In some embodiments, the agents described herein can be administered to a subject by any mode of administration that delivers the agent systemically or locally to a desired surface or target, and can include, but is not limited to, injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the agents described herein, other than directly into a target site, tissue, or organ, such that it enters the subject's circulatory system and, thus, is subject to metabolism and other like processes.

Efficacy

The efficacy of a composition in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved orameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example animal models of cancer, e.g. a murine xenograft model. When using an experimental animal model, efficacy of treatment is evidenced when a statistically significant change in a marker is observed.

In vitro and animal model assays are provided herein which allow the assessment of a given dose of a composition. The efficacy of a given dosage combination can also be assessed in an animal model, e.g. a murine xenograft model.

Some embodiments of the technology described herein can be defined according to any of the following numbered paragraphs:
1. A method of treating a disease or disorder involving inappropriate immunosuppression, the method comprising administering to a subject in need thereof, a therapeutically effective amount of an inhibitor of TIM-1 expression or activity in B cells, thereby treating the disease or disorder.
2. The method of paragraph 1, wherein the disease or disorder is selected from cancer and chronic infection.
3. The method of paragraph 1 or paragraph 2, wherein the inhibitor of TIM-1 is targeted to B cells.
4. The method of any one of paragraphs 1-3, wherein the B cells comprise Regulatory B cells (Bregs).
5. The method of any one of paragraphs 1-4, wherein the inhibitor of TIM-1 comprises a TIM-1 inhibitory moiety and a B cell targeting moiety.
6. The method of paragraph 5, wherein the TIM-1 inhibitory moiety is selected from the group consisting of an antibody or antigen-binding fragment thereof, a small molecule, a peptide or polypeptide, a nucleic acid, and a therapeutic virus.
7. The method of paragraph 5 or paragraph 6, wherein the TIM-1 inhibitory moiety comprises an antibody or antigen-binding domain thereof that specifically binds TIM-1.
8. The method of paragraph 6, wherein the nucleic acid is selected from the group consisting of an RNA interference (RNAi) molecule, a short interfering RNA (siRNA), a short hairpin
RNA (shRNA), a micro-RNA (miRNA), an aptamer and a CRISPR-Cas system.
9. The method of any one of claims 5-8, wherein the B cell targeting moiety comprises a moiety that specifically binds to a B cell-specific cell-surface polypeptide.
10. The method of paragraph 9, wherein the B cell-specific cell surface polypeptide is selected from the group consisting of CD19, CD20, and CD22.
11. The method of any one of paragraphs 5-10, wherein the B cell targeting moiety comprises an antibody or antigen-binding fragment thereof, an aptamer, or a natural ligand that specifically binds the B cell-specific cell surface polypeptide.
12. The method of any one of paragraphs 1-11, wherein the inhibitor of TIM-1 reduces expression or activity of one or more selected from the group of TIGIT, TIM-3, LAG3, CTLA4, PD-1, CD39, and CD73 in B cells.
13. The method of any one of paragraphs 1-12, further comprising administering a therapeutically effective amount of an inhibitor of the expression or activity of one or more immune checkpoint polypeptides.
14. The method of paragraph 13, wherein the one or more immune checkpoint polypeptides are selected from the group consisting of TIGIT, TIM-3, LAG3, CTLA4, and PD-1.
15. The method of any one of paragraphs 1-14, further comprising, administering an inhibitor of TIGIT expression or activity.
16. The method of paragraph 14 or paragraph 15, wherein the inhibitor of TIGIT expression or activity is targeted to B cells.
17. The method of any one of paragraphs 14-16, wherein the inhibitor of TIGIT expression or activity is selected from the group consisting of an antibody or antigen-binding fragment thereof, a small molecule, a peptide or polypeptide, a nucleic acid and a therapeutic virus.
18. The method of paragraph 17, wherein the antibody or antigen-binding fragment thereof comprises the CDRs of a TIGIT-specific antibody in Table 4.
19. The method of paragraph 17, wherein the nucleic acid is selected from the group consisting of
an RNA interference (RNAi) molecule, a short interfering RNA (siRNA), a short hairpin RNA (shRNA), a micro-RNA (miRNA), an aptamer, and a CRISPR-Cas system.
20. The method of any one of paragraphs 14-18, wherein the inhibitor of TIM1 and the inhibitor of TIGIT expression or activity are comprised by a multispecific inhibitory agent comprising an inhibitor of TIM-1 expression or activity and an inhibitor of TIGIT expression or activity.
21. The method of paragraph 20, wherein the multispecific inhibitory agent comprises one or more antibody antigen-binding domains.
22. A method of reducing B cell-mediated immunosuppression in a subject in need thereof, the method comprising administering to a subject in need thereof, a therapeutically effective amount of an inhibitor of TIM-1 expression or activity in B cells, thereby reducing B cell mediated immunosuppression in the subject.
23. The method of paragraph 22, wherein the inhibitor of TIM-1 is targeted to B cells.
24. The method of paragraph 22 or paragraph 23, wherein the B cells comprise Regulatory B cells (Bregs).
25. The method of any one of paragraphs 22-24, wherein the inhibitor of TIM-1 comprises a TIM-1 inhibitory moiety and a B cell targeting moiety.
26. The method of paragraph 25, wherein the TIM-1 inhibitory moiety is selected from the group consisting of an antibody or antigen-binding fragment thereof, a small molecule, a peptide or polypeptide, a nucleic acid and a therapeutic virus.
27. The method of paragraph 25 or paragraph 26, wherein the TIM-1 inhibitory moiety comprises an antibody or antigen-binding domain thereof that specifically binds TIM-1.
28. The method of paragraph 26, wherein the nucleic acid is selected from the group consisting of
an RNA interference (RNAi) molecule, a short interfering RNA (siRNA), a short hairpin RNA (shRNA), a micro-RNA (miRNA), an aptamer, and a CRISPR-Cas system.
29. The method of any one of paragraphs 25-28, wherein the B cell targeting moiety comprises a moiety that specifically binds to a B cell-specific cell-surface polypeptide.
30. The method of paragraph 29, wherein the B cell-specific cell surface polypeptide is selected from the group consisting of CD19, CD20 and CD22.
31. The method of any one of paragraphs 25-30, wherein the B cell targeting moiety comprises an antibody or antigen-binding fragment thereof, or an aptamer.
32. The method of any one of paragraphs 22-31, wherein the inhibitor of TIM-1 reduces expression or activity of one or more of TIGIT, TIM-3 LAG3, CTLA4 or PD-1 in Breg cells.
33. The method of any one of paragraphs 22-32, further comprising administering a therapeutically effective amount of an inhibitor of the expression or activity of one or more immune checkpoint polypeptides.
34. The method of paragraph 33, wherein the one or more immune checkpoint polypeptides are selected from the group consisting of TIGIT, TIM-3, LAG3, CTLA4, PD-1, CD39, and CD73.
35. The method of any one of paragraphs 22-32, further comprising administering an inhibitor of TIGIT expression or activity.
36. The method of paragraph 35, wherein the inhibitor of TIGIT expression or activity is selected from the group consisting of an antibody or antigen-binding fragment thereof, a small molecule, a peptide or polypeptide, a nucleic acid and a therapeutic virus.
37. The method of paragraph 36, wherein the antibody or antigen-binding fragment thereof comprises the CDRs of TIGIT-specific antibody in Table 4.
38. The method of paragraph 36, wherein the nucleic acid is selected from the group consisting of
an RNA interference (RNAi) molecule, a short interfering RNA (siRNA), a short hairpin RNA (shRNA), a micro-RNA (miRNA), an aptamer and a CRISPR-Cas system.
39. The method of any one of paragraphs 35-37, wherein the inhibitor of TIM1 and the inhibitor of TIGIT expression or activity are comprised by a multispecific inhibitory agent comprising an inhibitor of TIM-1 expression or activity and an inhibitor of TIGIT expression or activity.
40. The method of paragraph 39, wherein the multispecific inhibitory agent comprises one or more antibody antigen-binding domains.
41. A composition comprising an inhibitor of TIM-1 expression or activity that is targeted to B cells.
42. The composition of paragraph 41, wherein the inhibitor of TIM-1 comprises a moiety that inhibits the expression or activity of TIM-1 and a moiety that specifically binds a B cell-specific cell surface polypeptide.
43. The composition of paragraph 42, wherein the moiety that inhibits the expression or activity of TIM-1 is selected from the group consisting of an antibody or antigen-binding fragment thereof, a small molecule, a peptide or polypeptide, a nucleic acid and a therapeutic virus.
44. The composition of paragraph 43, wherein the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.
45. The composition of any one of paragraphs 42-44, wherein the B cell-specific cell surface polypeptide is selected from the group consisting of CD19, CD20, and CD22.
46. The composition of any one of paragraphs 42-45, wherein the moiety that specifically binds a B cell-specific cell surface polypeptide comprises an antibody or antigen-binding fragment thereof, or an aptamer.
47. The composition of any one of paragraphs 41-46, further comprising one or more inhibitors of the expression or activity of one or more immune checkpoint polypeptides.
48. The composition of paragraph 47, wherein the one or more immune checkpoint polypeptides comprises TIGIT.
49. The composition of paragraph 48, wherein the inhibitor of TIGIT expression or activity is selected from the group consisting of an antibody or antigen-binding fragment thereof, a small molecule, a peptide or polypeptide, a nucleic acid and a therapeutic virus.
50. The composition of paragraph 49, wherein the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.
51. The composition of paragraph 49, wherein the antibody or antigen-binding fragment thereof comprises the CDRs of TIGIT-specific antibody in Table 4.
52. A composition comprising an inhibitor of TIGIT expression or activity that is targeted to B cells.
53. The composition of paragraph 52, wherein the inhibitor of TIGIT expression or activity is selected from the group consisting of an antibody or antigen-binding fragment thereof, a small molecule, a peptide or polypeptide, a nucleic acid and a therapeutic virus.
54. The composition of paragraph 53, wherein the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.
55. The composition of paragraph 53, wherein the antibody or antigen-binding fragment thereof comprises the CDRs of a TIGIT-specific antibody in Table 4.
56. A method of treating a disease or disorder involving inappropriate immunosuppression, the method comprising administering to a subject in need thereof, a therapeutically effective amount of an inhibitor of TIM-1 expression or activity and a therapeutically effective amount of an inhibitor of PD-1 expression or activity, thereby treating the disease or disorder.
57. The method of paragraph 56, wherein the disease or disorder is selected from cancer and chronic infection.
58. The method of paragraph 56 or paragraph 57, wherein the administering comprises administering a multispecific inhibitory agent comprising a TIM-1 inhibitory moiety and a PD-1 inhibitory moiety.
59. The method of any one of paragraphs 56-58, wherein the inhibitor of TIM-1 is targeted to B cells.
60. The method of paragraph 59, wherein the B cells comprise Regulatory B cells (Bregs).
61. The method of any one of paragraphs 56-60, wherein the inhibitor of TIM-1 comprises a TIM-1 inhibitory moiety and a B cell targeting moiety.
62. The method of paragraph 61, wherein the TIM-1 inhibitory moiety is selected from the group consisting of an antibody or antigen-binding fragment thereof, a small molecule, a peptide or polypeptide, a nucleic acid, and a therapeutic virus.
63. The method of paragraph 61 or paragraph 62, wherein the TIM-1 inhibitory moiety comprises an antibody or antigen-binding domain thereof that specifically binds TIM-1.
64. The method of paragraph 62, wherein the nucleic acid is selected from the group consisting of
an RNA interference (RNAi) molecule, a short interfering RNA (siRNA), a short hairpin RNA (shRNA), a micro-RNA (miRNA), an aptamer and a CRISPR-Cas system.
65. The method of any one of paragraphs 61-64, wherein the B cell targeting moiety comprises a moiety that specifically binds to a B cell-specific cell-surface polypeptide.
66. The method of paragraph 65, wherein the B cell-specific cell surface polypeptide is selected from the group consisting of CD19, CD20, and CD22.
67. The method of paragraph 65 or paragraph 66, wherein the B cell targeting moiety comprises an antibody or antigen-binding fragment thereof, an aptamer, or a natural ligand that specifically binds the B cell-specific cell surface polypeptide.
68. The method of any one of paragraphs 56-67, wherein the inhibitor of TIM-1 reduces expression or activity of one or more selected from the group of TIGIT, TIM-3, LAG3, CTLA4, PD-1, CD39, and CD73 in B cells.
69. An inhibitor of TIM-1 expression or activity that is targeted to B cells, for use in the treatment of a disease or disorder involving inappropriate immunosuppression.
70. The inhibitor for use of paragraph 69, wherein the disease or disorder is cancer or chronic infection.
71. The inhibitor for use of paragraph 69 or paragraph 70, wherein the inhibitor of TIM-1 comprises a moiety that inhibits the expression or activity of TIM-1 and a moiety that specifically binds a B cell-specific cell surface polypeptide.
72. The inhibitor for use of paragraph 71, wherein the moiety that inhibits the expression or activity of TIM-1 is selected from the group consisting of an antibody or antigen-binding fragment thereof, a small molecule, a peptide or polypeptide, a nucleic acid and a therapeutic virus.
73. The inhibitor for use of paragraph 72, wherein the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.
74. The inhibitor for use of any one of paragraphs 71-73, wherein the B cell-specific cell surface polypeptide is selected from the group consisting of CD19, CD20, and CD22.
75. The inhibitor for use of any one of paragraphs 71-74, wherein the moiety that specifically binds a B cell-specific cell surface polypeptide comprises an antibody or antigen-binding fragment thereof, or an aptamer.
76. A composition comprising an inhibitor of TIM-1 expression or activity and an inhibitor of an immune checkpoint inhibitor polypeptide for use in the treatment of a disease or disorder involving inappropriate immunosuppression.
77. The composition for use of paragraph 76, wherein the disease or disorder is cancer or chronic infection.
78. The composition for use of paragraph 76 or paragraph 77, wherein the inhibitor of TIM-1 comprises a moiety that inhibits the expression or activity of TIM-1 and a moiety that specifically binds a B cell-specific cell surface polypeptide.
79. The composition for use of paragraph 78, wherein the moiety that inhibits the expression or activity of TIM-1 is selected from the group consisting of an antibody or antigen-binding fragment thereof, a small molecule, a peptide or polypeptide, a nucleic acid and a therapeutic virus.
80. The composition for use of paragraph 79, wherein the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.
81. The composition for use of any one of paragraphs 78-80, wherein the B cell-specific cell surface polypeptide is selected from the group consisting of CD19, CD20, and CD22.
82. The composition for use of any one of paragraphs 78-81, wherein the moiety that specifically binds a B cell-specific cell surface polypeptide comprises an antibody or antigen-binding fragment thereof, or an aptamer.
83. The composition for use of any one of paragraphs 76-82, wherein the one or more immune checkpoint polypeptides comprises TIGIT.
84. The composition for use of paragraph 83, wherein the inhibitor of TIGIT expression or activity is selected from the group consisting of an antibody or antigen-binding fragment thereof, a small molecule, a peptide or polypeptide, a nucleic acid and a therapeutic virus.
85. The composition for use of paragraph 84, wherein the nucleic acid is selected from the group consisting of an RNA interference (RNAi) molecule, a short interfering RNA (siRNA), a short hairpin RNA (shRNA), a micro-RNA (miRNA), an aptamer and a CRISPR-Cas system.
86. The composition of for use of paragraph 84, wherein the antibody or antigen-binding fragment thereof comprises the CDRs of a TIGIT-specific antibody in Table 4.

It is to be understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

The following examples are provided by way of illustration not limitation.

### Example 1: Role of of Tim-1+ B Cells in Regulating Tumor Growth

Tim-1 is a transmembrane glycoprotein expressed in several immune subsets and regulates their responses. It has been shown that a large majority of IL-10 producing B cells are Tim-1 positive (Tim-1+) B cells, regardless of other markers, and that transfer of Tim-1+ B cells inhibits experimental autoimmune encephalomyelitis (EAE), allograft rejection, and allergic airway inflammation. Mice with either global Tim-1 deficiency (Tim-1−/−) or harboring a loss of function Tim-1 mutant (Tim-1Δmucin) showed profound defects in B cell IL-10 production, and with age developed severe spontaneous multiorgan tissue inflammation. Young mice lacking TIM-1 expression specifically in B-cells (Tim-1BKO) develop more severe EAE upon induction which could not fully recover, compared to control mice. Their B cells produced more proinflammatory cytokines, and promoted Th1 and Th17 responses, but inhibited the generation of Foxp3+ Tregs and Tr1 cells. Mechanistically, Tim-1 as a phosphatidylserine receptor is required for optimal IL-10 production and function of Bregs by sensing apoptotic cells (AC), and Tim-1 expression in B cells is required for AC treatment-mediated inhibition of EAE. Importantly, it has been demonstrated that B cell IL-10 is enriched in Tim-1+ cells, which can suppress T cell responses, in various models of inflammatory settings and several human diseases. These data indicate that Tim-1 is necessary for Breg IL-10 production and immunosuppressive function in tolerance maintenance and inflammation restraint.

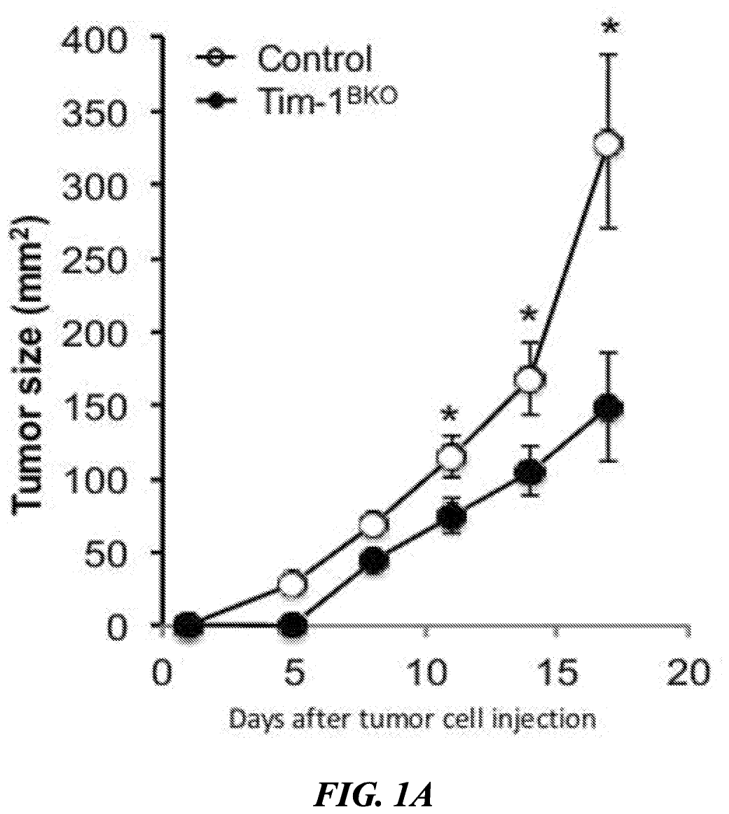

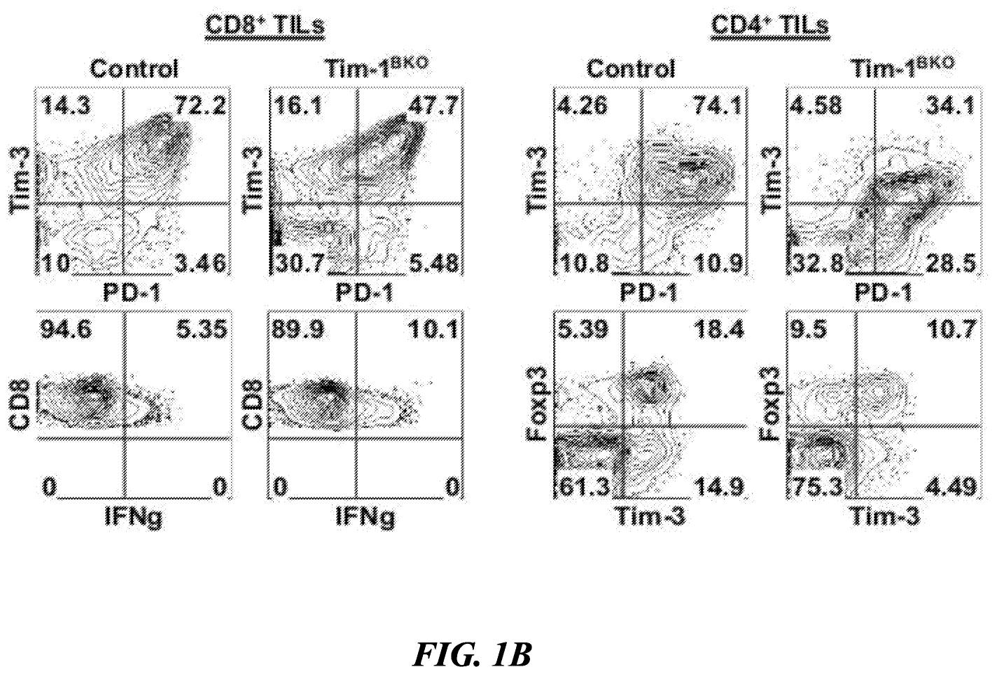

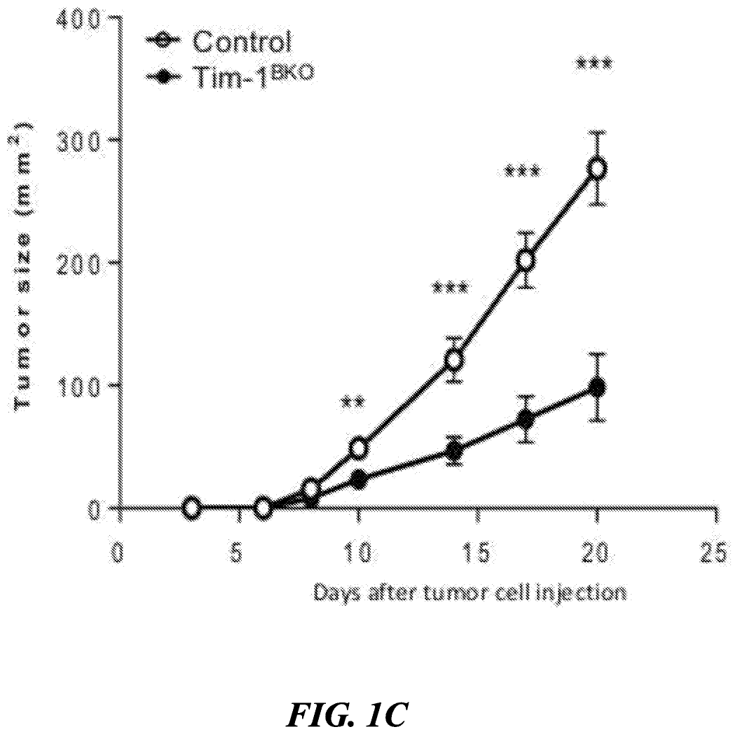

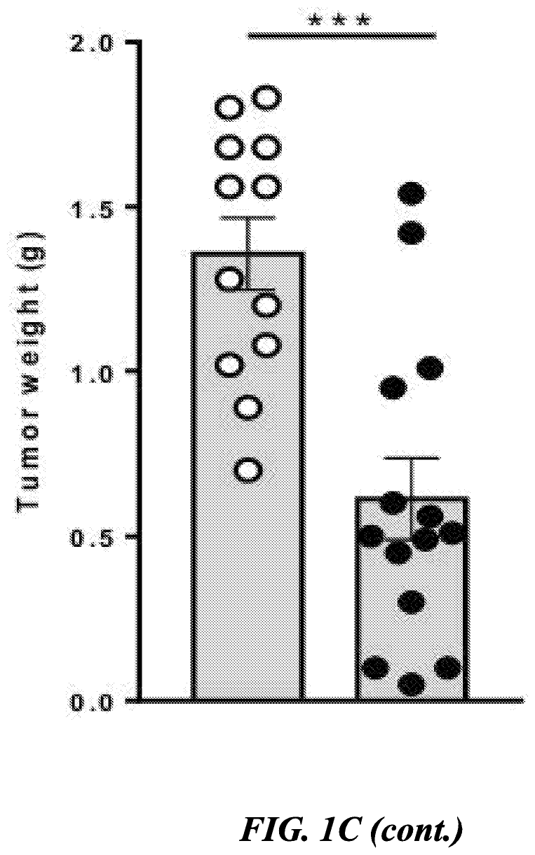

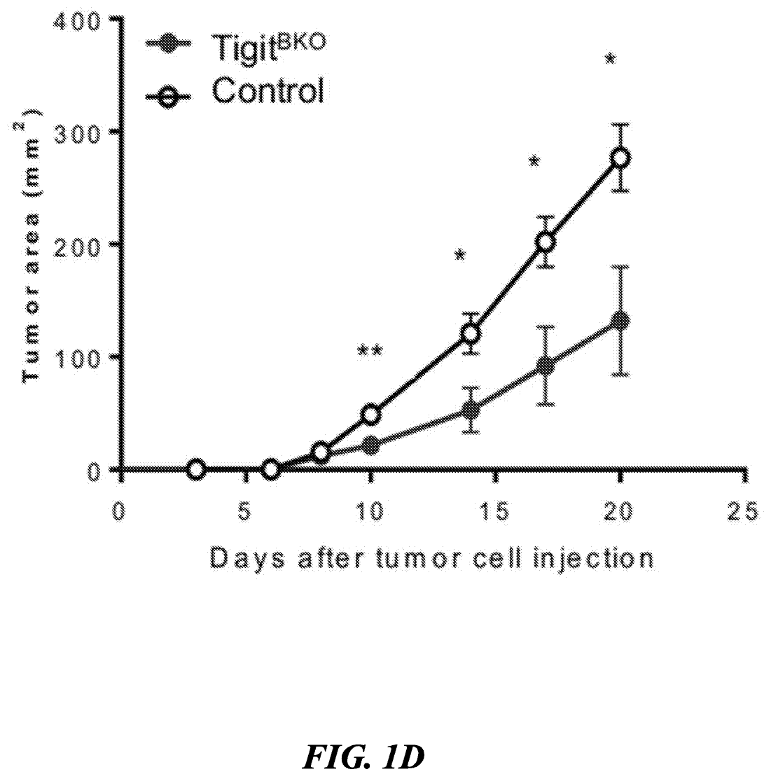

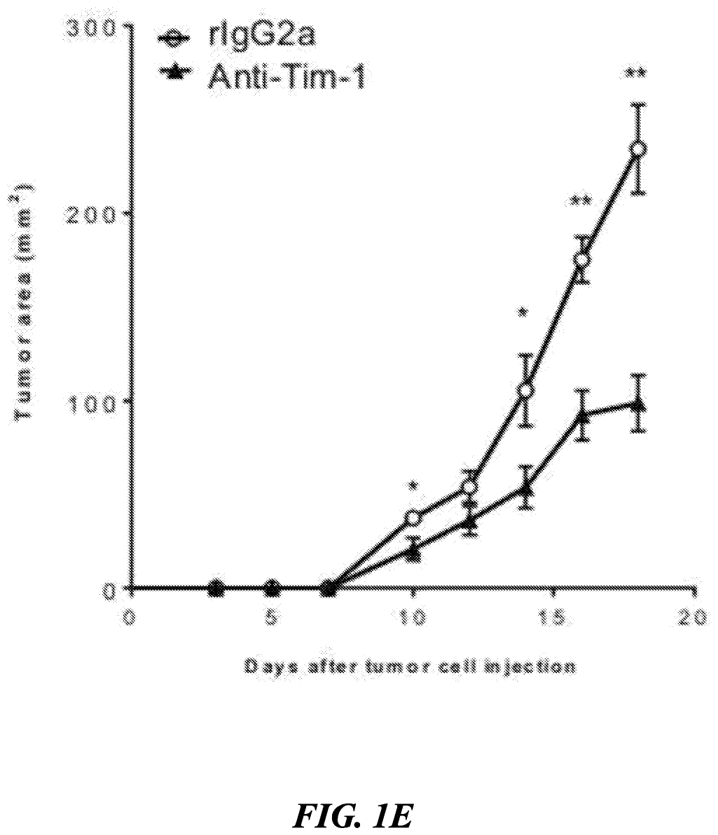

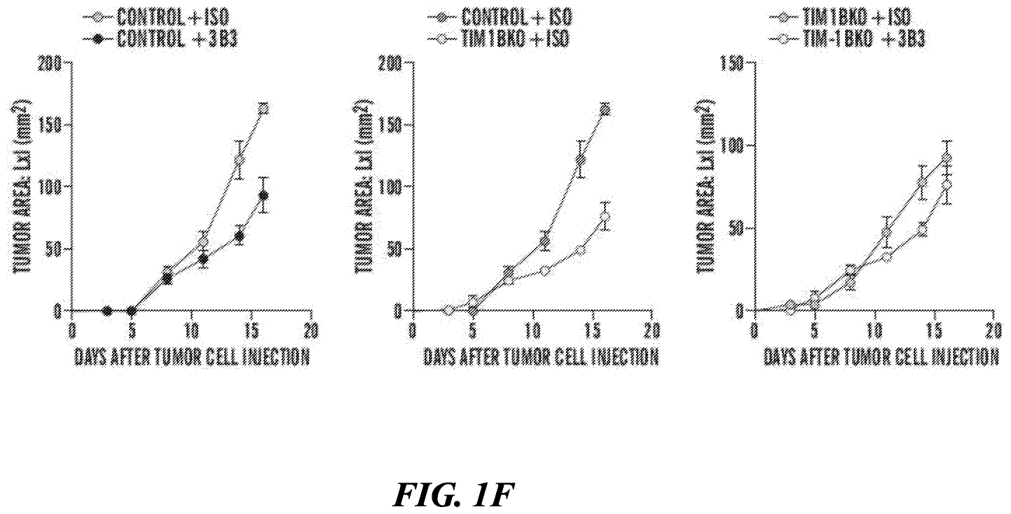

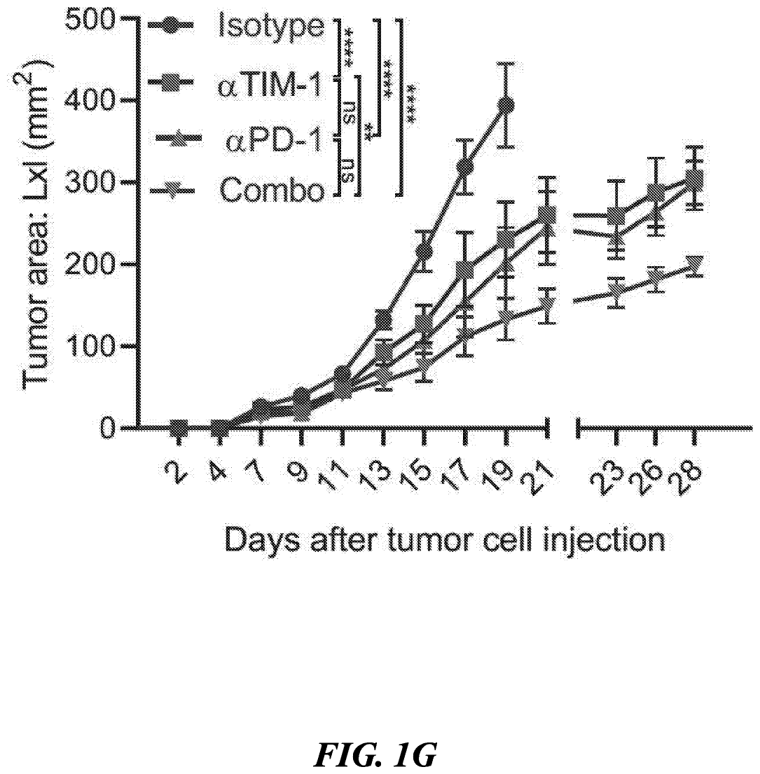

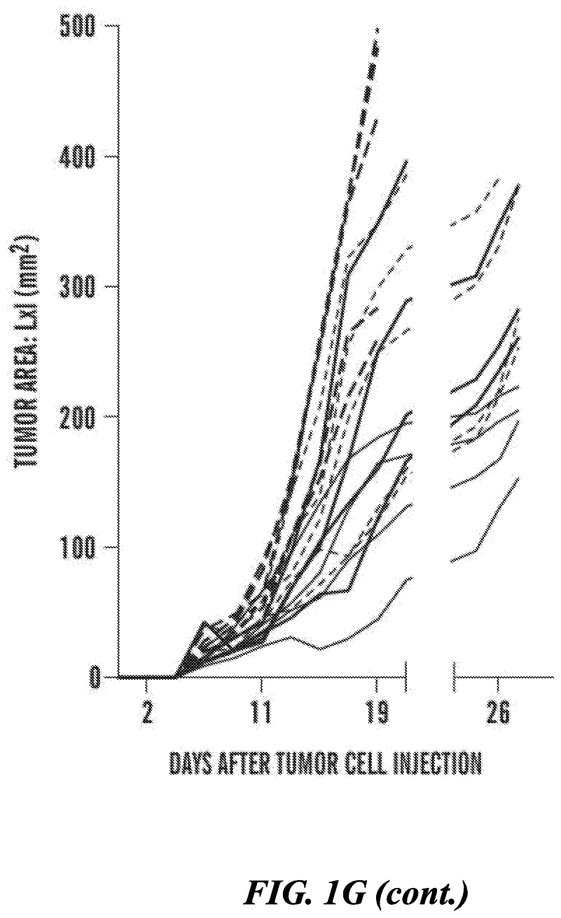

To clearly and faithfully demonstrate the role the role of Tim-1+ B cells in regulating tumor growth, several transplantable mouse tumors in WT and Tim-1BKO mice were tested. Colon adenocarcinoma MC38 cells were implanted subcutaneously in mice and it was found that MC38 growth was significantly restricted in Tim-1BKO mice, compared to that in control mice (FIG. 1A). Analysis of tumor infiltrating CD4+ and CD8+ T cells showed that MC38-bearing Tim-1BKO mice exhibited dramatically decreased frequency of exhausted PD1+ Tim-3+ cells in both CD4+ and CD8+ T cell populations and CD4+Foxp3+ Tregs, and an increased frequency of IFN-g producing CD8+ T cells (FIG. 1B).

These data indicate that Tim-1BKO mice with impaired Breg cells inhibit tumor growth by promoting effector T cells and decreasing Tregs. In addition, the growth of B16F10 melanoma tumors was reduced in Tim-1BKO mice (FIG. 1C).

Furthermore, it was demonstrated that in addition to IL-10, Tim-1+ B cells differentially express a set of co-inhibitory “check-point” receptors, including TIGIT, whose optimal expression was impaired in the absence of Tim-1 expression signaling. As demonstrated herein, TigitBKO mice not only display more severe EAE upon induction at young age, but with age also preferentially developed spontaneous paralysis with CNS inflammation. Moreover, it was shown that TigitBKO mice inhibited B16F10 growth (FIG. 1D), thus further supporting an important role of TIGIT in regulating B cell-mediated tumor growth. Consistent with this, WT mice treated with an anti-Tim-1 mAb (clone 3B3) showed a significantly reduced B16F10 growth (FIG. 1E).

Collectively, the data described herein indicate that targeting Tim-1+ B cells or their regulatory functions can be used for immunotherapeutic strategies in cancer.

### Example 2: Role of Inhibition of Both Tim-1 and its Coinhibitory Molecules Such as TIGIT in B Cells in Regulating Tumor Growth

Hosts with B-cell specific deletions of either TIM-1 or TIGIT strongly inhibit tumor development. Accordingly, an improvement in efficacy of treating cancer can be realized by inhibiting both TIM-1 and TIGIT in B cells in an individual suffering from cancer. As but one example, treatment of a cohort of patients can include a regimen of regular intravenous infusions of a polypeptide construct including antigen-binding domains specific for TIM-1 (e.g., an scFv including the CDRs of the anti-TIM-1 monoclonal antibody clone 3A12E10), TIGIT (e.g., an scFv including the CDRs of monoclonal antibody clone BMS-986207) and CD20 (e.g., an scFv including the CDRs of the Veltuzumab). Efficacy can be monitored relative to a cohort of subjects receiving anti-TIGIT therapy alone in both B cell-targeted and non-targeted forms, to determine improved efficacy in the subjects receiving the B-cell targeted anti TIM-1, anti-TIGIT therapy.

### Example 3: Sequences

TABLE 1
Antibodies specific for TIM-1.

| | | Reference |
|---|---|---|
| Target | Clone Name | Information |
| TIM-1 | 3A12E10 | Abcam |
| TIM-1 | 219211 | R&D systems |
| TIM-1 | A-12 | Santa Cruz |
| TIM-1 | 3B3 | Bio X cell |
| TIM-1 | 1.29 | U.S. 2005/0084449 |
| TIM-1 | 1.37 | U.S. 2005/0084449 |
| TIM-1 | 2.16 | U.S. 2005/0084449 |
| TIM-1 | 3B3 | U.S. 20150079085A1 |

Below are sequences related to monoclonal antibodies against TIM-1. With regard to the amino acid sequences, bold indicates framework regions, underlining indicates CDR regions, and italics indicates constant regions.

Anti-TIM-1 mAb 1.29
Nucleotide sequence of heavy chain variable
region and a portion of constant region (SEQ ID No. 8):
5′TGGGTCCTGTCCCAGGTGCAGCTGCAGGAGTCGGG
CCCAGGACTGGTGAAGCCTTCGGAGACCCTGTCCCTC.
ACCTGCACTGTCTCTGGTGGCTCCGTCAGCAGTGGTG
GTTACTACTGGAGCTGGATCCGGCAGCCCCCAGGGAA
GGGACTGGAGTGGATTGGGTTTATCTATTACACTGGG
AGCACCAACTACAACCCCTCCCTCAAGAGTCGAGTCT
CCATATCAGTAGACACGTCCAAGAACCAGTTCTCCCT
GAAGCTGAGCTCTGTGACCGCTGCGGACGCGGCCGTG
TATTACTGTGCGAGAGATTATGACTGGAGCTTCCACT
TTGACTACTGGGGCCAGGGAACCCTGGTCACCGTCTC
CTCAGCCTCCACCAAGGGCCCATCGGTCTTCCCCCTG
GCGCCCTGCTCCAGGAGCACCTCCGAGAGCACAGCGG
CCCTGGGCTGCCTGGTCAAGGACTACTTCCCCGAACC
GGTGACGGTGTCGTGGAACTCAGGCGCTCT3′
Amino acid sequence of heavy chain variable
region and a portion of constant region encoded by (SEQ ID
NO: 9):
WVLSQWQLQESGPGLVKPSETLSLTCTVSGGSVS
SGGYYWSWIRQPPGKGLEWIGFIYYTGSTNYNPS
LKSRWSISWDTSKNQFSLKLSSWTAADAAWYYCA
RDYDWSFHFDYWGQGTLWTWSSASTKGPSVFPLA
PCSRSSESTAAGCVKDYFPEPWTYVSWNSGA
Nucleotide sequence of light chain variable region
and a portion of constant region (SEQ ID No. 10):
5′ CAGCTCCTGGGGCTCCTGCTGCTCTGGTTCCCAGG
TGCCAGGTGTGACATCCAGATGACCCAGTCTCCATCC
TCCCTGTCTGCATCTATAGGAGACAGAGTCACCATCA
CTTGCCGGGCAAGTCAGGGCATTAGAAATGATTTAGG
CTGGTATCAGCAGAAACCAGGGAAAGCCCCTAAGCGC
CTGATCTATGCTGCATCCAGTTTGCAAAGTGGGGTCC
CATCAAGGTTCAGCGGCAGTGGATCTGGGACAGAATT
CACTCTCACAATCAGCAGCCTGCAGCCTGAAGATTTT
GCAACTTATTACTGTCTACAGCATAATAGTTACCCTC.
TCACTTTCGGCGGAGGGACCAAGGTGGAGATCAAACG
AACTGTGGCTGCACCATCTGTCTCATCTTCCCGCCA
TCTGATGAGCAGTTGAAATCTGGAACTGCCTCTGTTG
TGTGCCTGCTGAATAACTTCTATCCCAGAGAGGCCAA
AGTACAGTGGAAGGTGGATAACGCC3′
Amino acid sequence of light chain variable region
and a portion of constant region encoded by (SEQ ID NO: 11):
QLLGLLLLWFPGARCDIQMTQSPSSLSASIGDRW
TITCRASQGIRNDLGWYQQKPGKAPKRLIYAASS
LQSGVPSRFSGSGSGTEFTLTISSLQPEDFATYY
CLQHNSYPLTFGGGTKVEIKRTVAAPSVHFPPS
DEQLKSGTASVVCLLNNFYPREAKVQWKVDNA
Anti-TIM-1 mAb 1.37
Nucleotide sequence of heavy chain variable
region and a portion of constant region (SEQ ID No: 12):
5′CAGTGTGAGGTGCAGCTGGTGGAGTCTGGGGGAGGC
TTGGTCCAGCCTGGGGGGTCCCTGAGACTCTCCTGTGC
AGCCTCTGGATTCACCTTTACTAACTATTGGATGAGCTG
GGTCCGCCAGGCTCCAGGGAAGGGGCTGGAGTGGGTGG
CCAACATACAGCAAGATGGAAGTGAGAAATACTATGTG
GACTCTGTGAGGGGCCGATTCACCATCTCCAGAGACAA
CGCCAAGAACTCACTGTATCTGCAAATGAACAGCCTGA
GAGCCGAGGACTCGGCTGTGTATTACTGTGCGAGATGG
GACTACTGGGGCCAGGGAACCCTGGTCACCGTCCCTCA
GCCTCCACCAAGGGCCCATCGGTCTTCCCCCTGGCGCCC
TGCTCCAGGAGCACCTCCGAGAGCACAGCGGCCCTGGG
CTGCCTGGTCAAGGACTACTTCCCCGAACCGGTGAGCGG
TGTCGTGGAAC3′
Amino acid sequence of heavy chain variable
region and a portion of constant region encoded by (SEQ ID
NO:13):
QCEVQLVESGGGLVQPGGSLRLSCAASGFTFTNY
WMSWVRQAPGKGLEWVANIQQDGSEKYYVDSVRG
RFTISRDNAKNSLYLQMNSLRAEDSAVYYCARWD
YWGQGTLVTVSSASTKGPSVFPLAPCSRSTSEST
AALGCLVKDYFPEPVSGVVE
Nucleotide sequence of light chain variable region
and a portion of constant region (SEQ ID No: 14):
5′CTTCTGGGGCTGCTAATGCTCTGGGTCCCTGGATC
CAGTGGGGATATTGTGATGACCCAGACTCCACTCTCC
TCAACTGTCATCCTTGGACAGCCGGCCTCCATCTCCT
GCAGGTCTAGTCAAAGCCTCGTACACAGTGATGGAAA
CACCTACTTGAATTGGCTTCAGCAGAGGCCAGGCCAG
CCTCCAAGACTCCAATTTATATGATTTCTAACCGGT
TCTCTGGGGTCCCAGACAGATTCAGTGGCAGTGGGGC
AGGGACAGATTTCACACTGAAAATCAGCAGGGTGGAA
GCTGAGGATGTCGGGGTTTATTACTGCATGCAAGCTA
CAGAATCTCCTCAGACGTTCGGCCAAGGGACCAAGGT
GGAAATCAAACGAACTGTGGCTGCACCATCTGTCTTC
ATCTTCCCGCCATCTGATGAGCAGTTGAAATCTGGAA
GGGCCTCTGTTG3′
Amino acid sequence of light chain variable region
and a portion of constant region encoded by (SEQ ID NO: 15):
LLGLLMLWVPGSSGDIVMTQTPLSSTWILGQPAS
ISCRSSQSLVHSDGNTYLNWLQQRPGQPPRLLIY
MISNRFSGVPDRFSGSGAGTDFLKISRVEAEDV
GVYYCMQATESPQTFGQGTKVEIKRTVAAPSVFI
FPPSDEQLKSGRASV
Anti-TIM-1 mAb 2.16
Nucleotide sequence of heavy chain variable
region and a portion of constant region (SEQ ID No: 16):
5′ GAGCAGTCGGGGGGAGGCGTGGTAAAGCCTGGGGG
GTCTCTTAGACTCTCCTGGCAGCCTCTGGATTCACT
TTCAGTAACGCCTGGATGACCTGGGTCCGCCAGGCTC
CAGGGAAGGGGCTGGAGTGGGTTGGCCGTATTAAAAG
GAGAACTGATGGTGGGACAACAGACTACGCTGCACCC
GTGAAAGGCAGATTCACCATCTCAAGAGATGATTCAA
AAAACACGCTGTATCTGCAAATGAACAACCTGAAAAA
CGAGGACACAGCCGTGTATTACTGTACCTCAGTCGAT
AATGACGTGGACTACTGGGGCCAGGGAACCCTGGTCA,
CCGTCCCTCAGCTCCACCAAGGGCCCACCGTCTT
CCCCCTGGCGCCCTGCTCCAGGAGCACCTCCGAGAGC
ACAGCCGCCCTGGGCTGCCTGGTCAAGGACTACTTCC
CCGAACCGGTGACGGTGTCGTGGAACTCAGGCGCCCT
GACCAGCGGCGTGCACACCTTCCCGGCTGTCCTACAG
TCCTCAGGACTCT3′
Amino acid sequence of heavy chain variable
region and a portion of constant region encoded by (SEQ ID
NO: 17):
XXXXEQSGGGWWKPGGSLRLSCAASGFTFSNAWM
TWWRQAPGKGLEWVGRIKRRTDGGTTDVAAPVKG
RFTISRIDDSKNTLYLQMNNLKNEDTAWYYCTSV
DNDVDYWGGGTLWTVSSASTKGPSVFPLAPCSRS
TSESAAGCVKDYFPEPWTWSWNSGAISGWH
TFPAVLQSSGL
Nucleotide sequence of light chain variable region
and a portion of constant region (SEQ ID No: 18):
5′CTGACTCAGTCTCCACTCTCCCTGCCCGTCACCC
CTGGAGAGCCGGCCTCCATCTCCTGCAGGTCTAGTC
AGAGCCTCCTGCATAGTAATGGATACAACTATTTGG
ATTGGTACCTGCAGAAGCCAGGGCAGTCTCCACAGC
TCCTGATCTATTGGGTTCTAATCGGGCCTCCGGGG
TCCCTGACAGGTTCAGTGGCAGTGGATCAGGCACAG
ATTTTACACTGAAAATCAGCAGAGTGGAGGCTGAGG
ATATTGGTCTTTATTACTGCATGCAAGCTCTACAAA
CTCCGCTCACTTTCGGCGGAGGGACCAAGGTGGACA
TCAAACGAACTGTGGCTGCACCATCTGTCTTCATCT
TCCCGCCATCTGATGAGCAGTTGAAATCTGGAACTG
CCTCTGTTGTGGCCTGCTGAATAACTTCACCCA
GAGAGGCCAAAGTACAG3′
Amino acid sequence of light chain variable region
and a portion of constant region encoded by (SEQ ID NO: 19):
XXXLTQSPLSLPWTPGEPASISCRSSQSLLHSNG
YNYLDWYLQKPGQLLIYLGSNRASGVPDRFS
GSGSGTDFTLKISRWEAEDIGLYYCMQALQTPLT
FGGGTKWDIKRTVAAPSVFIFPPSDEQLKSGTAS
VVCLLNNFYPREAKVQ

TABLE 2
Exemplary RNAi sources for TIM-1

| Target | Name | Reference Information |
|---|---|---|
| TIM-1 | TIM-1 siRNA | Santa Cruz Biotechnology |
| TIM-1 | TIM-1 shRNA | Santa Cruz Biotechnology |
| TIM-1 | TIM-1 shRNA | Sigma-Aldrich |

TABLE 3
Exemplary Aptamer references for TIM-1

| Target | Name | Reference Information |
|---|---|---|
| TIM-1 | TIM-1 | Ebrahimi et al. Iranian Journal of |
| | aptamer | Biotechnology, 2015, Page 25-31. |

TABLE 4
TIGIT-specific antibodies

| Target | Antibody Name | Source |
|---|---|---|
| TIGIT | 13E7 | Novus Biologicals |
| TIGIT | MBSA43 | Thermo Fisher Scientific |
| TIGIT | BMS-986207 | Bristol-Myers Squibb |
| TIGIT | 2; 2C; 3; 5; 13; 13A; 13B; | WO2018160704 |
| | 13C; 13D; 14; 16; 16 C; | |
| | 16D; 16E; 18; 21; 22; 25; | |
| | 25A; 25B; 25C; 25D; | |
| | 25E; 27; 54 | |
| TIGIT | 10A7 | WO2015009856A2 |
| TIGIT | 14A6; 28H5; 31C6 | WO2016028656A1 |
| TIGIT | 1F4 | WO2016011264A1 |
| TIGIT | Ab6; Ab7; Ab12; Ab17; Ab18; | U.S. 20180155422 |
| | Ab19; Ab20; Ab23; Ab24; | |
| | Ab26; Ab29; Ab34; Ab36; | |
| | Ab39; Ab43; Ab51; Ab54; | |
| | Ab55; Ab58; Ab61; Ab64; | |
| | Ab65; Ab66; Ab69; Ab75; | |
| | Ab77; Ab80; Ab82; Ab83; | |
| | Ab84; Ab86; Ab91; Ab92; | |
| | Ab94; Ab95; Ab96; Ab98; | |
| | Ab100; Ab102; Ab 103; | |
| | Ab105; Ab106; Ab107; | |
| | Ab108; Ab109; Ab 110; | |
| | Ab114; Ab115; Ab116; | |
| | Ab117; Ab121; Ab122; | |
| | Ab123; Ab127; Ab130; | |
| | Ab132; Ab133; Ab134; | |
| | Ab135; Ab136; Ab138; | |
| | Ab140; Ab144; Ab145; | |
| | Ab147; Ab149; Ab150; | |
| | Ab151; Ab155; Ab158; | |
| | Ab159; Ab160; Ab161; | |
| | Ab162; Ab163; Ab164; | |
| | Ab165; Ab166; Ab168; | |
| | Ab171; Ab174; Ab 176; | |
| | Ab177; Ab178; Ab179; | |
| TIGIT | G1 D265A | WO2016106302A9 |
| | 4B1 | |
| TIGIT | TX99; TX100; TX103; | Nakamura Y et al., Monoclon |
| | TX104; TX105 | Antib Immunodiagn |
| | | Immunother. 2018. |
| TIGIT | 4.1D3 | U.S. 20180186875 |
| TIGIT | 14A6; 28H5; 3106 | U.S. 20180066055A1 |

TABLE 5
Antibodies specific for PD-1.

| Target | Clone Name | Reference Information |
|---|---|---|
| PD-1 | NAT105 | Abcam |
| PD-1 | RMP1-14 | BioCell |
| PD-1 | 5B7-E9-G2 | RayBiotech |
| PD-1 | RMP1-30 | MyBioSource.com |
| PD-1 | SPM597 | NSJ Bioreagents |
| PD-1 | 8A1 | Bioss |
| PD-1 | PD1.3 | U.S. 8,741,295 |
| PD-1 | hu317-4B6PD1 antibody | U.S. 10/519,235 |
| PD-1 | 67D9, c67D9, and hu67D9 | WO058115 |
| PD-1 | 949 | U.S. 0,229,461 |
| PD-1 | 90G12F8 | WO2019160755 |

Below are sequences related to monoclonal antibodies against PD-1.

Monoclonal Antibody 67D9 (see, e.g. WO058115):

Heavy chain CDR1:
(SEQ ID NO: 20)
Thr Tyr Gly Met Ser
Heavy chain CDR2:
(SEQ ID NO: 21)
Thr Ile Ser Gly Gly Gly Arg Asp Thr Tyr Tyr Pro Asp Thr Val Lys
Heavy chain CDR3
(SEQ ID NO: 22)
Gln Asp Tyr Gly Asn Tyr Val Trp Phe Ala Tyr
Light chain variable region comprising
Light chain CDR1:
(SEQ ID NO: 23)
Arg Ala Ser Glu Ser Val Asp Ser Tyr Gly Ile Ser Phe Met His
Light chain CDR2:
(SEQ ID NO: 24)
Ser Thr Ser Asn Arg Gly Ser
Light chain CDR3:
(SEQ ID NO: 25)
Gln Gln Ser Gln Glu Val Pro Trp Thr

Monoclonal Antibody 90G12F8 (see, e.g. WO2019160755):

The anti-PD-1 antibody or antigen-binding fragment thereof comprises

(a) light chain complementarity determining regions (CDRs) comprising a sequence of amino acids as set forth below and heavy chain CDRs comprising a sequence of amino acids as set forth below:

Light chain CDR1:
(SEQ ID NO: 26)
Arg Ala Ser Lys Gly Val Ser Thr Ser Gly Tyr Ser Tyr Leu His
Light chain CDR2:
(SEQ ID NO: 27)
Leu Ala Ser Tyr Leu Glu Ser
Light chain CDR3:
(SEQ ID NO: 28)
Gln His Ser Arg Asp Leu Pro Leu Thr
Heavy chain CDR1:
(SEQ ID NO: 29)
Asn Tyr Tyr Met Tyr
Heavy chain CDR2:
(SEQ ID NO: 30)
Gly Ile Asn Pro Ser Asn Gly Gly Thr Asn Phe Asn Glu Lys Phe Lys
Asn
Heavy chain CDR3:
(SEQ ID NO: 31)
Arg Asp Tyr Arg Phe Asp Met Gly Phe Asp Tyr
Light chain variable region:
(SEQ ID NO: 32)
Glu Ile Val Leu Thr Gln Ser Pro Ala Thr Leu Ser Leu Ser Pro Gly
Glu Arg Ala Thr Leu Ser Cys Arg Ala Ser Lys Gly Val Ser Thr Ser
Gly Tyr Ser Tyr Leu His Trp Tyr Gln Gln Lys Pro Gly Gln Ala Pro
Arg Leu Leu Ile Tyr Leu Ala Ser Tyr Leu Glu Ser Gly Val Pro Ala
Arg Phe Ser Gly Ser Gly Ser Gly Thr Asp Phe Thr Leu Thr Ile Ser
Ser Leu Glu Pro Glu Asp Phe Ala Val Tyr Tyr Cys Gln His Ser Arg
Asp Leu Pro Leu Thr Phe Gly Gly Gly Thr Lys Val Glu Ile Lys
Light chain:
(SEQ ID NO: 33)
Glu Ile Val Leu Thr Gln Ser Pro Ala Thr Leu Ser Leu Ser Pro Gly
Glu Arg Ala Thr Leu Ser Cys Arg Ala Ser Lys Gly Val Ser Thr Ser
Gly Tyr Ser Tyr Leu His Trp Tyr Gln Gln Lys Pro Gly Gln Ala Pro
Arg Leu Leu Ile Tyr Leu Ala Ser Tyr Leu Glu Ser Gly Val Pro Ala
Arg Phe Ser Gly Ser Gly Ser Gly Thr Asp Phe Thr Leu Thr Ile Ser
Ser Leu Glu Pro Glu Asp Phe Ala Val Tyr Tyr Cys Gln His Ser Arg
Asp Leu Pro Leu Thr Phe Gly Gly Gly Thr Lys Val Glu Ile Lys Arg
Thr Val Ala Ala Pro Ser Val Phe Ile Phe Pro Pro Ser Asp Glu Gln
Leu Lys Ser Gly Thr Ala Ser Val Val Cys Leu Leu Asn Asn Phe Tyr
Pro Arg Glu Ala Lys Val Gln Trp Lys Val Asp Asn Ala Leu Gln Ser
Gly Asn Ser Gln Glu Ser Val Thr Glu Gln Asp Ser Lys Asp Ser Thr
Tyr Ser Leu Ser Ser Thr Leu Thr Leu Ser Lys Ala Asp Tyr Glu Lys
His Lys Val Tyr Ala Cys Glu Val Thr His Gln Gly Leu Ser Ser Pro
Val Thr Lys Ser Phe Asn Arg Gly Glu Cys
Heavy chain variable region:
(SEQ ID NO: 34)
Gln Val Gln Leu Val Gln Ser Gly Val Glu Val Lys Lys Pro Gly Ala
Ser Val Lys Val Ser Cys Lys Ala Ser Gly Tyr Thr Phe Thr Asn Tyr
Tyr Met Tyr Trp Val Arg Gln Ala Pro Gly Gln Gly Leu Glu Trp Met
Gly Gly Ile Asn Pro Ser Asn Gly Gly Thr Asn Phe Asn Glu Lys Phe
Lys Asn Arg Val Thr Leu Thr Thr Asp Ser Ser Thr Thr Thr Ala Tyr
Met Glu Leu Lys Ser Leu Gln Phe Asp Asp Thr Ala Val Tyr Tyr Cys
Ala Arg Arg Asp Tyr Arg Phe Asp Met Gly Phe Asp Tyr Trp Gly Gln
Gly Thr Thr Val Thr Val Ser Ser
Heavy chain:
(SEQ ID NO: 35)
Gln Val Gln Leu Val Gln Ser Gly Val Glu Val Lys Lys Pro Gly Ala
Ser Val Lys Val Ser Cys Lys Ala Ser Gly Tyr Thr Phe Thr Asn Tyr
Tyr Met Tyr Trp Val Arg Gln Ala Pro Gly Gln Gly Leu Glu Trp Met
Gly Gly Ile Asn Pro Ser Asn Gly Gly Thr Asn Phe Asn Glu Lys Phe
Lys Asn Arg Val Thr Leu Thr Thr Asp Ser Ser Thr Thr Thr Ala Tyr
Met Glu Leu Lys Ser Leu Gln Phe Asp Asp Thr Ala Val Tyr Tyr Cys
Ala Arg Arg Asp Tyr Arg Phe Asp Met Gly Phe Asp Tyr Trp Gly Gln
Gly Thr Thr Val Thr Val Ser Ser Ala Ser Thr Lys Gly Pro Ser Val
Phe Pro Leu Ala Pro Cys Ser Arg Ser Thr Ser Glu Ser Thr Ala Ala
Leu Gly Cys Leu Val Lys Asp Tyr Phe Pro Glu Pro Val Thr Val Ser
Trp Asn Ser Gly Ala Leu Thr Ser Gly Val His Thr Phe Pro Ala Val
Leu Gln Ser Ser Gly Leu Tyr Ser Leu Ser Ser Val Val Thr Val Pro
Ser Ser Ser Leu Gly Thr Lys Thr Tyr Thr Cys Asn Val Asp His Lys
Pro Ser Asn Thr Lys Val Asp Lys Arg Val Glu Ser Lys Tyr Gly Pro
Pro Cys Pro Pro Cys Pro Ala Pro Glu Phe Leu Gly Gly Pro Ser Val
Phe Leu Phe Pro Pro Lys Pro Lys Asp Thr Leu Met Ile Ser Arg Thr
Pro Glu Val Thr Cys Val Val Val Asp Val Ser Gln Glu Asp Pro Glu
Val Gln Phe Asn Trp Tyr Val Asp Gly Val Glu Val His Asn Ala Lys
Thr Lys Pro Arg Glu Glu Gln Phe Asn Ser Thr Tyr Arg Val Val Ser
Val Leu Thr Val Leu His Gln Asp Trp Leu Asn Gly Lys Glu Tyr Lys
Cys Lys Val Ser Asn Lys Gly Leu Pro Ser Ser Ile Glu Lys Thr Ile
Ser Lys Ala Lys Gly Gln Pro Arg Glu Pro Gln Val Tyr Thr Leu Pro
Pro Ser Gln Glu Glu Met Thr Lys Asn Gln Val Ser Leu Thr Cys Leu
Val Lys Gly Phe Tyr Pro Ser Asp Ile Ala Val Glu Trp Glu Ser Asn
Gly Gln Pro Glu Asn Asn Tyr Lys Thr Thr Pro Pro Val Leu Asp Ser
Asp Gly Ser Phe Phe Leu Tyr Ser Arg Leu Thr Val Asp Lys Ser Arg
Trp Gln Glu Gly Asn Val Phe Ser Cys Ser Val Met His Glu Ala Leu
His Asn His Tyr Thr Gln Lys Ser Leu Ser Leu Ser Leu Gly Lys

TABLE 2
Exemplary RNAi sources for PD-1

| Target | Name | Reference Information |
|---|---|---|
| PD-1 | PD-1 siRNA | Invitrogen |
| PD-1 | PD-1 shRNA | Sigma-Aldrich |

TABLE 3
Exemplary Aptamer references for TIM-1

| Target | Name | Reference Information |
|---|---|---|
| PD-1 | PD-1 aptamer | Biochimie February 2018; 145: 125-130. |

TABLE 5
Antibodies specific for PD-L1.

| Target | Clone Name | Reference Information |
|---|---|---|
| PD-L1 | 28-8 | Abcam |
| PD-L1 | CD-274 | Thermo Fisher Scientific |
| PD-L1 | H-130 | Santa Cruz |
| PD-L1 | MEDI4736 | WO2015181331 |

Monoclonal Antibody MEDI4736 (see, e.g. WO2015181331):

MED 14736 and antigen-binding fragments thereof comprises a light chain variable region comprising the amino acid sequence of SEQ ID NO: 36 and a heavy chain variable region comprising the amino acid sequence of SEQ ID NO: 37. In a particular embodiment, MED 14736 or an antigen-binding fragment thereof for use in the methods provided herein comprises a heavy chain variable region and a light chain variable region, wherein the heavy chain variable region comprises the CDR1, CDR2, and CDR3 sequences of SEQ ID NOS: 38, 39, and 40, respectively, and wherein the light chain variable region comprises the CDR1, CDR2, and CDR3 sequences of SEQ ID NOS: 41, 42, and 43, respectively.

light chain variable region:
(SEQ ID NO: 36)
Glu Ile Val Leu Thr Gln Ser Pro Gly Thr Leu Ser Leu Ser Pro Gly
Glu Arg Ala Thr Leu Ser Cys Arg Ala Ser Gln Arg Val Ser Ser Ser
Tyr Leu Ala Trp Tyr Gln Gln Lys Pro Gly Gln Ala Pro Arg Leu Leu
Ile Tyr Asp Ala Ser Ser Arg Ala Thr Gly Ile Pro Asp Arg Phe Ser
Gly Ser Gly Ser Gly Thr Asp Phe Thr Leu Thr Ile Ser Arg Leu Glu
Pro Glu Asp Phe Ala Val Tyr Tyr Cys Gln Gln Tyr Gly Ser Leu Pro
Trp Thr Phe Gly Gln Gly Thr Lys Val Glu Ile Lys
Heavy chain variable region:
(SEQ ID NO: 37)
Glu Val Gln Leu Val Glu Ser Gly Gly Gly Leu Val Gln Pro Gly Gly
Ser Leu Arg Leu Ser Cys Ala Ala Ser Gly Phe Thr Phe Ser Arg Tyr
Trp Met Ser Trp Val Arg Gln Ala Pro Gly Lys Gly Leu Glu Trp Val
Ala Asn Ile Lys Gln Asp Gly Ser Glu Lys Tyr Tyr Val Asp Ser Val
Lys Gly Arg Phe Thr Ile Ser Arg Asp Asn Ala Lys Asn Ser Leu Tyr
Leu Gln Met Asn Ser Leu Arg Ala Glu Asp Thr Ala Val Tyr Tyr Cys
Ala Arg Glu Gly Gly Trp Phe Gly Glu Leu Ala Phe Asp Tyr Trp Gly
Gln Gly Thr Leu Val Thr Val Ser Ser
Heavy chain CDR1:
(SEQ ID NO: 38)
Arg Tyr Trp Met Ser
Heavy chain CDR2:
(SEQ ID NO: 39)
Asn Ile Lys Gln Asp Gly Ser Glu Lys Tyr Tyr Val Asp Ser Val Lys Gly
Heavy chain CDR3:
(SEQ ID NO: 40)
Glu Gly Gly Trp Phe Gly Glu Leu Ala Phe Asp Tyr
Light chain CDR1:
(SEQ ID NO: 41)
Arg Ala Ser Gln Arg Val Ser Ser Ser Tyr Leu Ala
Light chain CDR2:
(SEQ ID NO: 42)
Asp Ala Ser Ser Arg Ala Thr
Light chain CDR3:
(SEQ ID NO: 43)
Gln Gln Tyr Gly Ser Leu Pro Trp Thr

### Notes:

Clone 13E7: mouse monoclonal; reacts with human, mouse
Clone MBSA43: mouse/IgG1, reactivity: human, mouse
done 10A7: reactive against both mouse and human TIGIT
WO2016106302A9: clones 15A6, 22G2, 11G11 or 10D7 are human or humanized antibodies.
WO2018160704 clones listed in table bind to human TIGIT.

### Example 4: TIM-1 Pharmacological Targeting Regulates Tumor Growth and Synergize with Anti-PD1 Immunotherapy

CD19CrexTIM-1fl/fl (TIM-1BKO) and CD19Cre (control) mice were implanted with B16F10 melanoma, and treated with anti-TIM-1 Ab (250 μg, clone 3B3) or isotype control on days 7, 9 and 11 and tumor growth was monitored (FIG. 1F). Reduced tumor growth was found upon anti-Tim-1 treatment that was abolished in absence of Tim-1-expressing B cells. This result shows a modulation of Tim-1+ B cells function using anti-Tim-1 immunotherapy. the efficacy of combining anti-Tim-1 and anti-PD-1 therapy was evaluated (FIG. 1G). C57B16/J (WT) mice were implanted with B16F10 melanoma and treated with anti-TIM-1 as well as with three doses of anti-PD1 (200 μg, clone RMP1-14) antibody or isotype control (FIG. 1G). Combined Tim-1 and PD-1 blockade significantly reduced B16F10 control tumor growth. Thus, these data show that blockade of Tim-1 function enhances antitumor responses to contemporary immune checkpoint blockade which depends on Tim-1+ B cells function.

1. A method of treating a disease or disorder involving inappropriate immunosuppression, the method comprising administering to a subject in need thereof, a therapeutically effective amount of an inhibitor of TIM-1 expression or activity in B cells, thereby treating the disease or disorder.

2. The method of claim 1, wherein the disease or disorder is selected from cancer and chronic infection.

3. The method of claim 1, wherein the inhibitor of TIM-1 is targeted to B cells.

4. The method of claim 3, wherein the B cells comprise Regulatory B cells (Bregs).

5. The method of claim 1, wherein the inhibitor of TIM-1 comprises a TIM-1 inhibitory moiety and a B cell targeting moiety.

6. The method of claim 5, wherein the TIM-1 inhibitory moiety is selected from the group consisting of an antibody or antigen-binding fragment thereof, a small molecule, a peptide or polypeptide, a nucleic acid, and a therapeutic virus.

7. (canceled)

8. The method of claim 6, wherein the nucleic acid is selected from the group consisting of: an RNA interference (RNAi) molecule, a short interfering RNA (siRNA), a short hairpin RNA (shRNA), a micro-RNA (miRNA), an aptamer and a CRISPR-Cas system.

9. The method of claim 5, wherein the B cell targeting moiety comprises a moiety that specifically binds to a B cell-specific cell-surface polypeptide.

10. The method of claim 9, wherein the B cell-specific cell surface polypeptide is selected from the group consisting of CD19, CD20, and CD22.

11. The method of claim 5, wherein the B cell targeting moiety comprises an antibody or antigen-binding fragment thereof, an aptamer, or a natural ligand that specifically binds the B cell-specific cell surface polypeptide.

12. (canceled)

13. The method of claim 1, further comprising administering a therapeutically effective amount of an inhibitor of the expression or activity of one or more immune checkpoint polypeptides.

14. The method of claim 13, wherein the one or more immune checkpoint polypeptides are selected from the group consisting of TIGIT, TIM-3, LAG3, CTLA4, and PD-1.

15. The method of claim 1, further comprising, administering an inhibitor of TIGIT expression or activity, optionally wherein the inhibitor of TIGIT expression or activity is targeted to B cells.

16. The method of claim 14, wherein the inhibitor of TIGIT expression or activity is targeted to B cells.

17.-19. (canceled)

20. The method of claim 14, wherein the inhibitor of TIM1 and the inhibitor of TIGIT expression or activity are comprised by a multispecific inhibitory agent comprising an inhibitor of TIM-1 expression or activity and an inhibitor of TIGIT expression or activity.

21. (canceled)

22. A method of reducing B cell-mediated immunosuppression in a subject in need thereof, the method comprising administering to a subject in need thereof, a therapeutically effective amount of an inhibitor of TIM-1 expression or activity in B cells, thereby reducing B cell mediated immunosuppression in the subject.

23.-32. (canceled)

33. The method of claim 22, further comprising administering a therapeutically effective amount of an inhibitor of the expression or activity of one or more immune checkpoint polypeptides.

34. (canceled)

35. The method of claim 22, further comprising administering an inhibitor of TIGIT expression or activity.

36.-38. (canceled)

39. The method of claim 35, wherein the inhibitor of TIM1 and the inhibitor of TIGIT expression or activity are comprised by a multispecific inhibitory agent comprising an inhibitor of TIM-1 expression or activity and an inhibitor of TIGIT expression or activity.

40. (canceled)

41. A composition comprising an inhibitor of TIM-1 expression or activity that is targeted to B cells.

42. The composition of claim 41, wherein the inhibitor of TIM-1 comprises a moiety that inhibits the expression or activity of TIM-1 and a moiety that specifically binds a B cell-specific cell surface polypeptide.

43.-86. (canceled)
